(19) United States
(12) Patent Application Publication
(10) Pub. No.: US 2023/0395224 A1
(43) Pub. Date: Dec. 7, 2023

(54) USER HEALTHCARE DATA ACCESSABILITY

(71) Applicant: Apple Inc., Cupertino, CA (US)

(72) Inventors: Jessica Pease, Mountain View, CA (US); Jim H. Qin, San Francisco, CA (US); Zhe Li, Santa Monica, CA (US)

(73) Assignee: Apple Inc., Cupertino, CA (US)

(21) Appl. No.: 18/205,887

(22) Filed: Jun. 5, 2023

Related U.S. Application Data
(60) Provisional application No. 63/349,535, filed on Jun. 6, 2022.

(51) Int. Cl. G16H 20/10; G16H 40/67

(52) U.S. Cl. CPC G16H 20/10; G16H 40/67; A61B 5/4833

(57) ABSTRACT

Aspects of the disclosure relate to medication calendaring. An example method can include a computing device receiving a first interval of days associated with a medication identifier and a second interval of days associated with the medication identifier, wherein the second interval of days sequentially follows the first interval of days. The computing device can determine a medication time interval, the medication time interval comprising the first interval of days and the second interval of days. The computing device can generate a second instance of the medication identifier. The computing device can calendar the second instance of the medication identifier on an electronic calendar via a sliding generation window, the sliding generation window comprising a consecutive set of days of the days of the medication period.

### CROSS-REFERENCE TO RELATED APPLICATIONS

This application is claims priority under 35 U.S.C. § 119(e) to U.S. Provisional Application No. 63/349,535, filed on Jun. 6, 2022, the contents of which is herein incorporated by reference.

### BACKGROUND

Advancements in cloud computing have allowed software applications to be tailored to each user's individual preferences and experiences. Users can expect to use their devices to access information from a cloud computing server in a private and secure manner. Health applications provide users with a unique experience of charting their individual health experiences. Given the number of aspects that health covers, user software solutions can be tailored to be responsive to individual aspects while maintaining the security of the user's information.

### BRIEF DESCRIPTION OF THE DRAWINGS

FIG. 1 is an illustration of ontology sharding between a user device and a server, in accordance with some embodiments.

FIG. 2 is a signaling diagram for ontology sharding, in accordance with some embodiments.

FIG. 3 is a process flow for server-side ontology sharding, in accordance with some embodiments.

FIG. 4 is a process flow for device-side ontology sharding, in accordance with some embodiments.

FIG. 5 is an illustration of a user domain concept (UDC) node creation node, in accordance with some embodiments.

FIG. 6 is a process flow for a UDC node creation, in accordance with some embodiments.

FIG. 7 is an illustration of a medication interaction detection process, in accordance with some embodiments.

FIG. 8 is a process flow for a medication interaction detection process, in accordance with some embodiments.

FIG. 9 is an illustration of a medication calendaring system, in accordance with some embodiments.

FIG. 10 is a process flow for medication calendaring, in accordance with some embodiments.

FIG. 11 is an illustration of a system for a health application, in accordance with some embodiments.

FIG. 12 is an illustration of a computing architecture, in accordance with some embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Mobile health applications have given users the ability to organize and manage important health-related information in a central and secure place. Health applications can provide current and accurate health-related information efficiently (in terms of device memory and CPU as well as device storage) while maintaining that user's privacy and recognizing the storage capacity of user devices.

A health application allows a user to search for and interact with health-related information in a privacy protecting manner that also efficiently uses compute power and storage. The application locally stores an ontology of relevant information on a user's device so that operations can be fully performed on the user's device.

The ontology can be a directed graph that includes health-related topics and associated information from various sources, such as medical journals, scientific studies, governmental reports, etc. Clinical health data is made more accessible to users through the use of this reference ontology. The ontology maps properties of medical record samples to particular medical record concepts using industry medical coding standards. One issue of performing operations associated with the ontology solely on a user's device, however, is providing the ability to update the ontology to add new relevant information and delete obsolete information while maintaining the user's privacy and respecting the user device's storage capacity, memory, and CPU.

There are clinical health features that include user configuration and customization (e.g., nicknaming a medication, adding a medication to an active-medications list, customizing a list of clinical labs to watch, etc.) These features assume user interactions with the data can persist, sync, and be shared. These features also assume that these concepts will remain stable and identifiable across operating system versions, ontology versions, and shares to other devices. Rather than mix user customization data into the reference ontology, and to avoid significant problems of syncing, handling different versions, and handling devices without reference ontologies, a health application may use user domain concepts (UDCs). Generally, a UDC may be a configurable abstraction around a health concept (e.g., a health concept identified from the health data) or an abstraction around such health concepts. Each UDC may have some amount of instance specific user configuration state (e.g., favorited, nickname, present in a given list, etc.). A UDC can be connected to underlying health data and can be synced and restored. For example, a smartphone application may enable a user to customize certain aspects of their health data that have been downloaded to the user's smartphone, and these customizations may be saved using user domain concepts. The UDCs can be stored locally on a device as nodes in a node-based relational data structure. The UDCs provide a structure for storing and maintaining customizable data that can be associated with reference concepts included in an ontology and user-specific data such as medical records, data collected from the user by a sensor, use-inputted data, etc. One issue for UDCs is to provide the ability for an application on a user's device to recognize associations between embedded ontology reference concepts and user-specific data, and different UDCs.

In addition to providing representations of medications and the reference ontology, in some implementations, a health application provides a user with the ability to determine whether medications may have potential interactions with each other. A health application can prompt a user to keep a medication list is kept up to date and show interactions with over-the-counter medications of which a user's healthcare professional may be unaware, as well as to show interactions with common lifestyle factors not often considered as medications.

Therefore, an application can be configured to compare prescribed medications to each other and to aspects of the user's lifestyle. The application can further alert the user to the possibility of an interaction between a combination of medications or a combination of a medication and a lifestyle aspect. Each comparison can be based on local information stored on the user's device and additionally can be performed locally on the user's device. One issue is that given the multitude of possible medications that may need to be cross-referenced, simply storing a list of each possible medication adverse interactions in the user's device would be overly burdensome on the device's memory system.

In addition to helping a user understand medication interactions, a health application may assist a user in scheduling medications. For example, a user device can assist with calendaring a user's medication regimen. A user can input medication and a schedule for the medication into a user device. For example, a health professional can direct a user to take the medication daily, weekly, or some other period. The user can enlist the application to help set up a medication calendar by inputting the medication and the medication's cycle (e.g., daily, weekly or another period) into the user device. The application can determine the user's medication schedule based on the cycle.

One aspect of the application can be calculating a schedule when a user initiates the application for medication calendaring after the medication regimen has begun. Another aspect of the application can be calculating a medication schedule when the medication includes more than one cycle, such that the user does not always take the medication at the same frequency. For example, a health professional can direct the user to take the medication for a term (e.g., one year). However, during the term, the instructions to take a particular medication may be to take the medication every day for the first twenty-three days of a month and then take no medication the last seven to eight days of the month. This pattern may be continued, for example, for a year. The user device needs to reconcile these medication instructions when generating a medication calendar.

The herein embodiments relate to applications in the health space. The embodiments further address one or more of the above-referenced issues while continuing to maintain the objectives of providing current and accurate information in a manner useful to a user, maintaining that user's privacy, and recognizing the storage capacity of user devices and speed and efficiency for device operations.

### Ontology Sharding

A reference ontology may include information about medications, vaccines, educational content for labs, or other medical related information. At any given time, a user of a device does not need the entire ontology on the device since the full ontology may take up a lot of storage space. Instead of storing an entire ontology, ontology sharding is a methodology for implementing and maintaining a relevant portion of the global reference ontology on a user's device. With sharding, a health application uses finer grained rules, or explicit feature opt-in triggers, to determine which individual pieces (shards) are required from the ontology.

A shard is an output of a selection criteria (a filter) against the full global ontology. A user can access their user device, to enter, request, or maintain their health information. When a user interacts with a health application, the interaction can trigger a fetch of a particular shard of the ontology. For example, if a user wants to add a medication to a list of medications that the user is taking, the device needs the portion of the ontology that includes information about the medication. This portion of the ontology is the medications feature search shard. The shard is required temporarily after the health application launch after updating the health application to include the medication feature. The shard is also required if the user enters the medication experience in the health application. When the user performs an operation that triggers the need for the medications feature search shard, the device first checks the embedded ontology for the required shard. If the shard does not exist on the device, the device then queries the global ontology for the specific shard.

Other feature triggers include (but are not limited to): a clinical health records trigger which requires the clinical health records feature shard when the user has at least one traditional clinical health record account on device or has one signed clinical data issuer account; a pill bottle scanning trigger that requires a pill bottle scanning shard when a user enters the pill bottle scanning experience in the health application; a medication interaction trigger which requires an interaction shard when the user enters the ‘add medication” experience or the device has two or more ontology supported mediation or one ontology supported medication and at least one enabled drug interaction lifestyle factor; a medication education trigger which requires the medication education shard when the user enters the “add medication” experience or the device has at least one ontology supported medication.

In addition to obtaining shards of the ontology-based on user directed interaction triggers, the device may obtain or update shards because of underlying condition triggers, including clinical health record updates.

The user device can access an embedded ontology that includes a hierarchical structure of nodes, in which each node includes information related to a health aspect, and nodes are connected to each other based on a relationship between their underlying health aspects. The user device can further retrieve information from the embedded ontology based on user input and present the information to the user.

For example, a user may add a medication to be tracked in a health application. The full ontology may include all available information about the medication (e.g., the normalized name of the medication, the preferred name, an identifier, and feature content). However, a user device may not need all of the information. If a user is adding the medication, the user may need to be able to identify the medication using the preferred name of the medication or the normalized name. Therefore, a shard would include the identifier, the normalized name, and the preferred name, but not the feature content. If a user wants to know the drug interactions, the user may need to know the feature content but not the preferred drug name or the normalized name, so a shard for drug-to-drug interactions may only include the feature content and the identifier and no naming information. In order to track a shard over time, a shard should have a stable identifier that can be used to identify the shard.

From time to time, an embedded ontology stored on the user's device may need to be updated to reflect a current state of health information. For example, a current embedded ontology may need to be updated to, for example, add new nodes, delete nodes, or reconfigure the connections between nodes based on new health-related information. Currently, the procedure for updating the current embedded ontology version on a device is for a server to transmit a global ontology to the user's device. Upon receipt of the global ontology, the device can install the current global ontology and delete the previous global ontology from the device.

This process of storing and updating the global ontology is encumbered with several inefficiencies. For example, the global ontology can require a large portion of disk space for storage. Furthermore, in the future, the amount of information provided by the global ontology will increase. As a result, more memory will be required to store the global ontology. Furthermore, in many instances updating a global ontology version can be based on incremental changes in the health space. In other words, the difference in information provided by a current version of the global ontology and a previous version of the global ontology can be relatively small. Yet, in order to preserve the privacy of the device holder, the updating process covers the entire global ontology rather than targeting updated portions. Furthermore, in addition to memory space, the amount of processing resources required to delete an older global ontology and install a new global ontology is not trivial.

Embodiments described herein address the above-referenced issues through a process of ontology sharding, wherein ontology shards of an embedded ontology stored on the user's device can be synchronized with ontology shards of a global ontology stored on a server. On the server-side, a global ontology can be formatted into a set of individual ontology shards, which are individual partitions of the global ontology. Each ontology shard can be identified with at least one feature that is associated with the data stored at the shard. Furthermore, when the server is engaging in an updating process, the server can transmit the identified ontology shard to be integrated with the embedded ontology on the user device, rather than the complete global ontology. When a new shard file is imported, a particular element (node, attribute, relationship can replace a persisted element of the ontology. The determination of whether to proceed with the replacement should seek to reduce the number of replacements (especially when an element value does not change) as ontology modifications can lead to downstream work, such as concept indexing, and can cause wear on a memory device, such as a flash storage device.

When an ontology element is created in the global ontology (or an inactive or active status is modified), the created ontology element can be tagged with a current global ontology version. The global ontology version can be incremented to correspond to publications of the global ontology so that ontology elements that are added or modified between versions can be tagged with the same global ontology version. This assists with tracing a particular ontology element to a particular ontology publication.

Once an entity is published, the only mutable fields for the entity are its status (active or inactive) and version (incrementing integer). However, an entity's logical definition can change due to the addition or inactivation of new child elements. For example:
a node can change its preferred name for a locale; and
and interaction node (described further below) can change its consumer-facing educational text and require a medication node (described further below) to refresh older content with newer content.

Entity versions are independent and unrelated to modifications or additions of child elements. In the event that ontology elements are exported from the global ontology in a shard, all the elements that meet the selection criteria can also be included. These exported elements include “active” nodes and predicate sets (i.e., sets of attributes and/or relationships for the concept nodes defined by a tuple of (source concept ID, attribute/relationship type). If a node, or all elements of the predicate set for a given node, have been marked “inactive,” then the node or predicate set can be represented in the export by some form of null-flag. In this sense, a removal can be handled without having to include all inactive elements in each shard publication.

On the device side, the user's device can store one or more individual data shards to form an embedded ontology. Additionally, during an updating process, the device can update individual ontology shards rather than the entire embedded ontology. By storing an embedded ontology rather than a global ontology, the herein described embodiments can offer reduced memory storage requirements for the user's device. Another benefit is that receiving and installing one or more ontology shards, rather than a global ontology, is less processing resource-intensive.

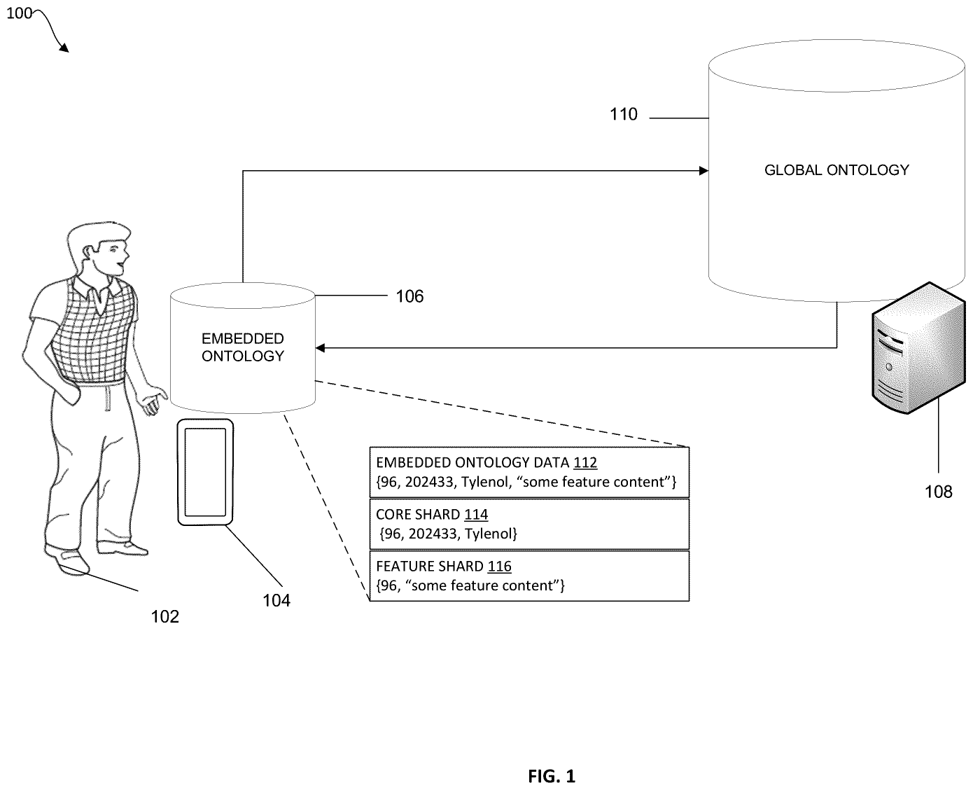

FIG. 1 is an illustration 100 of ontology sharding between a user device and a server, in accordance with some embodiments. As illustrated, a user 102 can have a user device 104, such as a smartphone, that stores one or more ontology shards that form an embedded ontology 106. Each ontology shard can include a stable identifier and data related to health. The data can be organized in a data structure, such as a directed graph, including a system of nodes and edges which are searchable by the user device. Each stable identifier can include a unique identifier for an ontology shard. In some embodiments, the data stored at one ontology shard can overlap with data stored at another ontology shard. In other words, more than one shard can include the same data. However, regardless of the content, each ontology shard has a unique stable identifier.

The embedded ontology 106 can be configured with respect to aspects of a health application loaded onto the user device. For example, the health application can include features directed toward one or more user medications. Furthermore, each of these features can include one or more sub-features, such as a medication identifier number, a preferred name for the medication, and feature content related to the medication. The embedded ontology 106 can include ontology shards that provide information relating to each of these features.

The user device 104 can be in communication with a server 108, which stores a global ontology 110. The global ontology 110 can include a complete set of ontology shards, which can each be a partition of data related to a health feature. Each ontology shard can include one or more ontology elements (e.g., nodes) that include information relating to a health feature. Each node can further be a parent node that includes one or more child nodes that are ontology elements that are connected to the parent node via an edge. Each node can further be identified by a unique node identifier.

Through a feature-based partitioning of the database into chunks of data, the global ontology 110 is divided into multiple shards. The shards can include a core shard and feature shards. The shards can be arranged such that a core shard includes ontology elements relating to core health concepts. The feature shards include ontology elements relating to a feature of the core health concepts. Each shard has a shard identifier that can be used to track a shard. A shard identifier includes a stable identifier that, along with schema type and schema version, form a globally unique identifier for a given shard and can be used to fetch a shard from the server. Ideally, these identifiers can be mostly human readable (for ease of debugging) and can be known prior to an operating system release in order to support mapping operating system features to particular shards required to support the feature.

A shard can include one or more versions, and the shard can be versioned for a particular stable identifier. A shard can be versioned based on a schema format. A shard file format can be easily fetched from the server and persist to disk without a significant impact on a daemon memory footprint. The shard file format can allow for enumeration/streaming from an on-disk representation into the embedded ontology to maintain a low memory footprint. In some embodiments, the shard file format can support the enumeration of only updated ontology elements to allow for faster importation/application of small difference updates. An example of embedded ontology data 112 that is related to a medication and stored in the embedded ontology 106 is provided in FIG. 1 for reference. The embedded ontology 106 can include, for example, a node identifier for identifying a node of the embedded ontology 106 (e.g., 96), a medication identifier number (e.g., 202433), a preferred name for the medication (e.g., Tylenol), and feature content (illustrated in FIG. 1 as “some feature content”.) The feature content can include data providing more feature-based information on a medication. Continuing with the Tylenol example, the feature content can include, for example, a picture of a Tylenol capsule, a published article on Tylenol, or other Tylenol-related information. Within the embedded ontology 106, some of these elements can be located in a core shard 114 and some other elements can be located in a feature shard 116. The core shard 114 can include, for example, the node identifier, the medication identifier, and the preferred name. The feature shard 116 can include, for example, node identifier, and the feature content.

As illustrated, a shard from the global ontology 110 can be exported by the server 108 to the user device 104 based on an input received from the user device 104. The input can include selection criteria that relate to an ontology shard of the global ontology 110. In some examples, the input may relate to a feature (e.g., a user adding a new medication for tracking in the health application), and based on this input, the user device 104 may generate the selection criteria relating to the new medication. Based on the selection criteria, the server 108 can identify one or more ontology shards (e.g., based on whether data in one ontology shard overlaps with data on another ontology shard). The server 108 can retrieve the one or more ontology shards and transmit them back to the user device 104. The user device 104 can download the one or more ontology shards and install them into the embedded ontology 106. Once installed, the user device 104 may retrieve medication-related information from the one or more shards that are now stored in the embedded ontology 106.

Each shard in the global ontology 110 can be stored as more than one version of the shard. Although different versions of a shard can include the same data, the shard schemas can be formatted to be compatible with different versions of an operating system of the user device 104. For example, each time that the user device's 104 operating system is updated, a new shard can be generated that is formatted to be compatible with the new version of the operating system. Consistent with a service provider's policies, shards formatted for older version(s) of an operating system can be deleted from the server 108. Therefore, in addition to the requested content, the selection criteria can include an operating system format for the user device 104.

As privacy regulations across the globe are more of a patchworked quilt than a uniform system, the server 108 can further take into consideration location of the user device. A patient in one location can be permitted, by the law of the location, to receive certain health information. The same user in another location can be prohibited, by the law of the location, from receiving the health information. The selection criteria can further include a location code identifying a location of the user device 104. Based on the location code, the server 108 can approve or reject a request for an ontology shard based on the location code.

From time to time, a shard stored on the server 108 can be updated, which can include adding new data, modifying old data, or deleting old data. The updating can be based on receiving new information or a modification of information. Updating an ontology shard can result in adding a new node, modifying a connection between two nodes, or deflecting a node. Each time that a node of an ontology shard is modified, it can be incrementally tagged to correspond to a version of the node. Therefore, if the user device 104 receives an ontology shard comprised of nodes, the user device 104 can determine whether each node is the same as a pre-existing node of the embedded ontology 106, or a new node that needs to be integrated into the embedded ontology 106. The tag can also be incremented to correspond to new publications of the global ontology 110.

The server 108 can further include an application programming interface (API) and a suite of importing software algorithms for importing information to populate the nodes of the global ontology 110. The API can identify a shard class based on a shard schema and map a shard to an importing software algorithm based on the schema. Identifying the class can further include periodic checks of a server state, a server observation, identifying an API for importation, and marking shards for eviction.

The server 108 can update a schema of an ontology element to support the sharding process. The schema update can be an update to the incremental version of the node as described above. The schema update can further be to update a shard identifier to identifier each shard that includes the node.

A shard registry can be stored on the user device 104 and associated with the health profiles of the user device 104. The shard registry can include a stable identifier for each shard stored in the embedded ontology. The stable identifier can include a schema type and a schema version for each shard. The stable element can be human readable and known version to a new release of an operating system version. The shard registry can include a desired state of each shard (e.g., active or inactive), a timestamp of the desired state, a current version, a current version of the data, a current region for the shard, a current local of the shard, a uniform resource locator (URL) of each shard, a URL timestamp, and a checksum for an available shard.

In the instance that an ontology shard of the global ontology 110 is updated at the server 108, the corresponding shard at the embedded ontology 106 may also need to be updated based on a previous interaction with the user device 104. In some embodiments, the selection criteria received from the user device 104 for retrieving an ontology shard can persist in the server 108. For example, a user device 104 can transmit a request to the server 108 for information related to cholesterol medication. At some point, after the server 108 services the request for the ontology shard that includes information related to the cholesterol medication, the ontology shard is updated at the server 108. The server 108 can store the previously received selection criteria for information related to cholesterol medication. Based on an update of the ontology shard that includes the information related to cholesterol medication, the server 108 can transmit the updated ontology shard to the user device 104, and without receiving a new request for information related to cholesterol medication from the user device.

On the device side, ontology elements (e.g., nodes) of a shard can be imported from the server 108 to the user device 104 to provide the user with medication related information. The user device 104 can receive an input from a user that triggers a request for the information. The trigger can be, for example, a creation of a UDC subclass, a medication entered into a medication calendar, a request for determination of a medication interaction, and any other suitable trigger as described herein. Based on the trigger, the user device 104 can search the embedded ontology 106 for information related to the input. In many instances, the embedded ontology 106 includes one or more pieces of information related to the input. For example, if the user enters the term “Altorvastatin”, the user device 104 can search the embedded ontology 106 for information related to this medication. As stated above, in many instances the embedded ontology 106 includes one or more pieces of information (e.g., brand names, images, ingredients). The user device 104 can retrieve the information from the embedded ontology 106 for presentation to the user.

In other instances, the embedded ontology 106 does not include information related to the input. For example, an input is related to a medication that was released after a last update of the embedded ontology 106. In these instances, the user device 104 can transmit a request to the server 108 for information related to the input, based on the embedded ontology not including the information. The request can include selection criteria that can be used to filter results from the global ontology 110.

In response to the request, the user device 104 can receive a response from the server 108 that includes an ontology shard from the global ontology 110. The user device 104 can install the ontology shard into the embedded ontology 106 one node at a time. The version number of each node in the embedded ontology 106 can be compared to the version number of the nodes of the imported ontology shard. If the imported node has a lower number than the node in the embedded ontology 106, the node in the embedded ontology 106 remains the same. If, however, the imported node has a higher version number than the node in the embedded ontology 106, the node in the embedded ontology 106 can be replaced with the imported node.

Once the user device 104 has replaced the previous ontology shard with the imported ontology shard, the user device can retrieve information from the ontology shard and display it to the user.

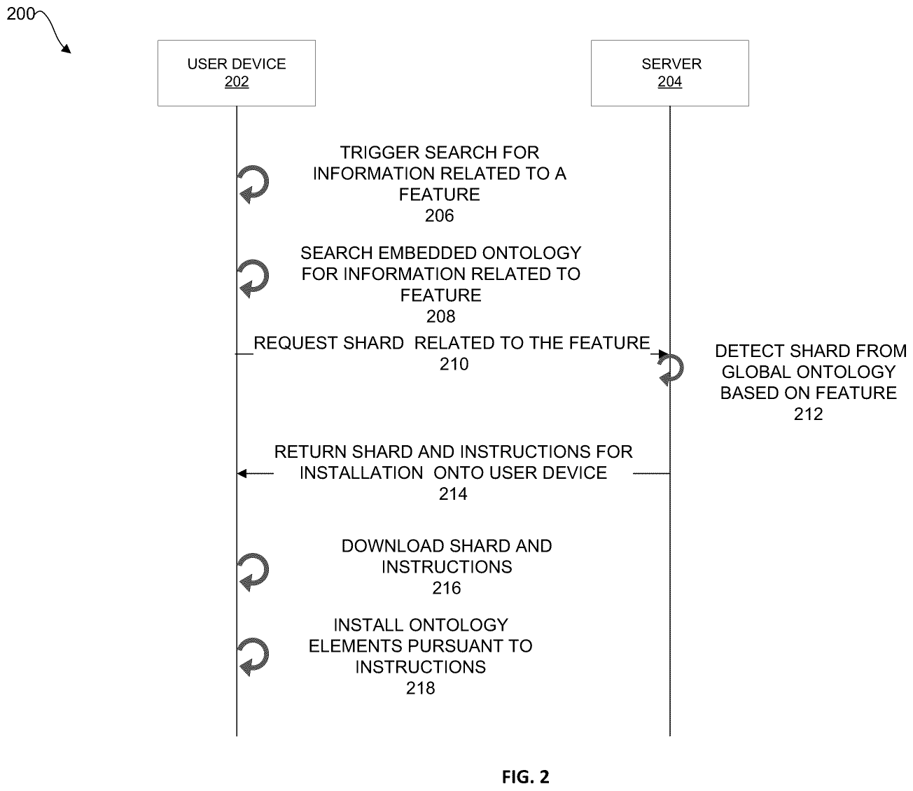

FIG. 2 is a signaling diagram 200 for ontology sharding, in accordance with some embodiments. As illustrated, a user device 202 can be in communication with a server 204. The user device 202 is an example of the user device 104 and the server 204 is an example of the server 108. While the operations of processes 200, 300, 400, 600, 700, 800, and 1000 are described as being performed by generic computers, it should be understood that any suitable device (e.g., a reader device, a responder device) may be used to perform one or more operations of these processes. Processes 200, 300, 400, 600, 700, 800, and 1000 (described below) are respectively illustrated as logical flow diagrams, each operation of which represents a sequence of operations that can be implemented in hardware, computer instructions, or a combination thereof. In the context of computer instructions, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

At 206, the user device 202 can detect a trigger for a search for information related to a medication. The trigger can be, for example, a search query entered into the user device 202. The user device 202 can further analyze the search request to determine a feature associated with the search query.

At 208, the user device 202 can search an embedded ontology stored on the user device 202. If the user device 202 locates the information in the embedded ontology, the user device 202 can retrieve the information for presentation to a user and the process 200 can conclude. If the user device 202 determines that the embedded ontology does not include an information related to the feature the process 200 can move to step 210. In other instances, even if the user device 202 find a shard that includes information related to the feature, the shard needs to be updated, or potentially a shard element related to the feature needs to be updated. In these instances, the process 200 also continues to step 210.

At 210, the user device 202 can transmit a request for an ontology shard related to the search query to the server 204. The request can include the feature related to the

At 212, the server 204 can use the feature as a filter to detect a shard related to the feature in a global ontology.

At 214, the server 204 can return the shard, including any instructions for installing the shard to the user device 202.

At 216, the user device 202 can download the shard and the instructions for installing the shard.

At 218, the user device 202 can install the shard pursuant to the instructions. In some embodiments, the user device 202 can install the shard, node by node. Each ontology element can include a tag that designates a version number of the node. The user device 202 can compare a node version number of the node received from the global ontology to the node version number of a comparable node of an embedded ontology on the user device 202. If the node received from the global ontology has a higher version number than the comparable node, the user device 202 can replace the node of the embedded ontology with the received node. If, however, the node received from the global ontology has an equal or lower version number than the comparable node, the user device 202 does not replace the comparable ontology element with the received ontology element.

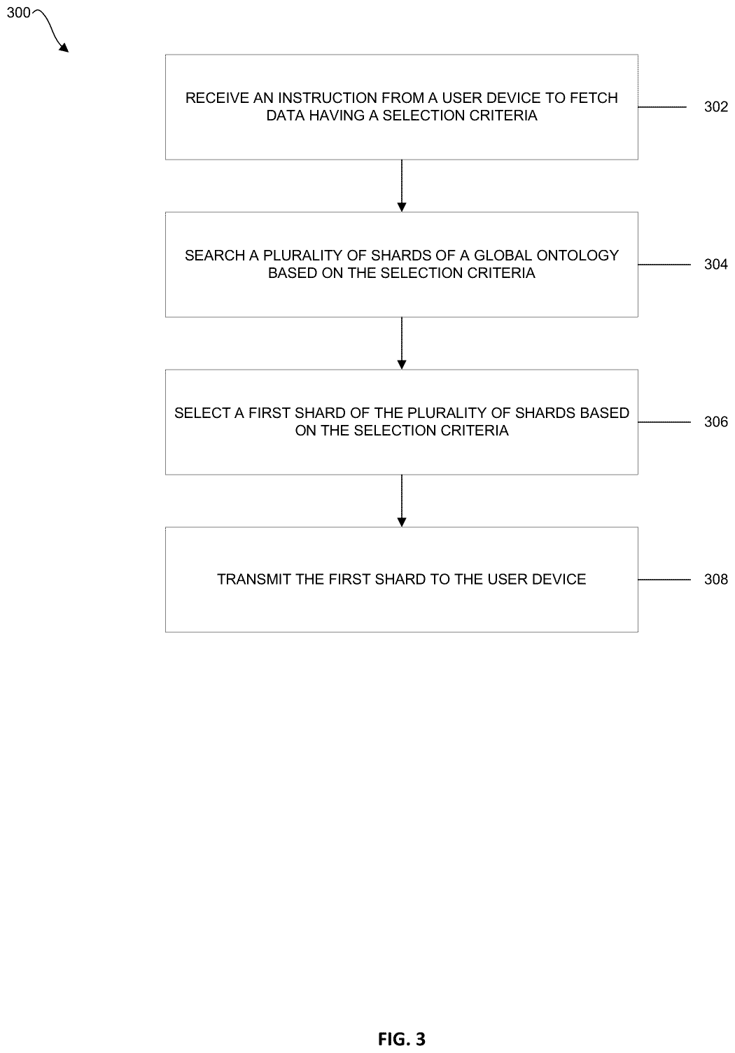

FIG. 3 is a process flow 300 for server-side ontology sharding, in accordance with some embodiments. At 302, a server such as the server 108, can receive an instruction to fetch data having a first selection criteria that the server 108 can use to filter the global ontology. For example, the selection criteria can relate to information regarding all medications that have a particular feature (e.g., an ingredient).

At 304, the server can search a plurality of shards of the global ontology based on the selection criteria. For example, the server can use the selection criteria as a filter to locate a shard that includes information related to the selection criteria.

At 306, the server can select a first shard based on the selection criteria. Using the selection criteria as a filter, the server can identify the first shard as having information related to the selection criteria. Using the example above, the selection criteria can include information regarding all medications that have a particular feature.

At 308, the server can transmit the first shard to the user device. The transmission can include computer-readable instructions for installing the first shard onto the user device. In some instances, the first shard is transmitted to the user device without a pre-existing embedded ontology. In these instances, the instructions may include steps for the user device to install the first shard onto the user device, including provisioning memory for an embedded ontology. In other instances, the first shard may be transmitted to a user device with a pre-existing embedded ontology. In these instances, the instructions may include steps for integrating the first shard into the pre-existing embedded ontology.

In some embodiments, the shard is transmitted based on a location of the user device 202 device. For example, the request can include a geographical region of the user device 202. In the event that data is restricted based on the geographical region of the user device 202.

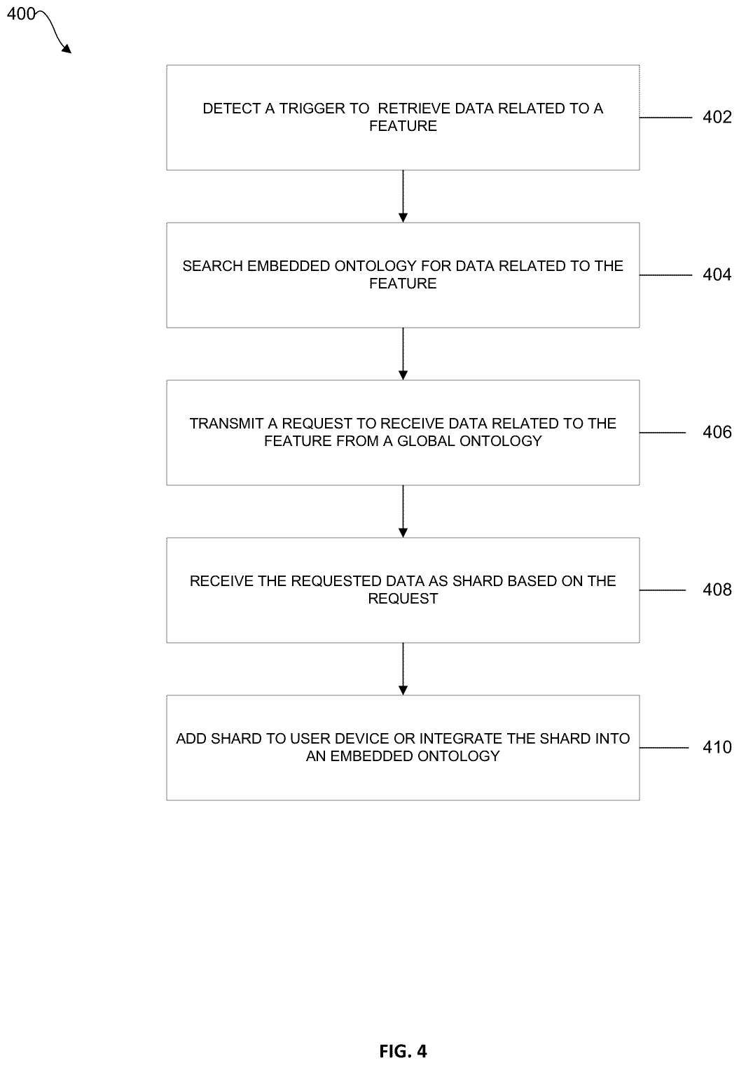

FIG. 4 is a process flow 400 for device-side ontology sharding, in accordance with some embodiments. At 402, a user device, (e.g., smartphone, laptop, and any other suitable device such as user device 104), can detect a trigger to retrieve data related to a feature.

At 404, the user device can search an embedded ontology for data related to the feature.

At 406, the user device can transmit a request to a server to receive data related to the feature in response to not finding data related to the feature in the embedded ontology. As described above, if the user device locates data in the embedded ontology related to the feature, it can retrieve the data for presentation to a user.

At 408, the user device can receive the requested data as a shard from the server based on the request. The user device can further receive computer-readable instructions for either add the shard on the user device, where the user device does not include a pre-existing embedded ontology or integrating the ontology shard into pre-existing embedded ontology.

At 410, the user device can add the shard to the user device or integrate the ontology shard into the embedded ontology as described above. In some embodiments, the receipt of certain data is restructured based on a geographical region, in which the user device is located. In these embodiments, the user device can have transmitted a geographical region of the user device. Furthermore, the integration of the ontology shard can be based on being in a geographical region that permits receipt of the data included in the ontology shard.

### User Domain Concepts

A user device (e.g., user device 104) can include a health application providing information regarding health concepts and the personal health information of a user. In many instances, a user's control over their health decisions can be empowered through information related to their personal data. For example, a user that has diabetes can input their diabetes medication into a user device to retrieve information related to the medication. It can be informative for the user to receive information to improve their diabetic condition. Information related to diabetes medication, including industry medical coding standards may be provided to the user in an embedded ontology. However, the user may want to customize the medication including, for example, nicknaming the medication and adding the medication to an active medication list. There is, of course, value in keeping the information included in the embedded ontology separate from the information included in the personal health information of the user.

Rather than integrate a customization of medication information into the embedded ontology, and to avoid issues related to synchronization of devices, and for devices that do not include embedded ontologies, a user domain concept (UDC) can be utilized. As introduced previously, a UDC is a user configurable abstraction related to a health concept. A UDC can be modeled as Base Schema+Connections to Samples+Connections to other UDCs+(optional connections to reference ontology)+Payload. The UDC can be represented as a node in a directed graph stored on a user device (e.g., user device 104). The use of UDCs permits the movement of a shard of one embedded ontology from a user profile to another user profile.

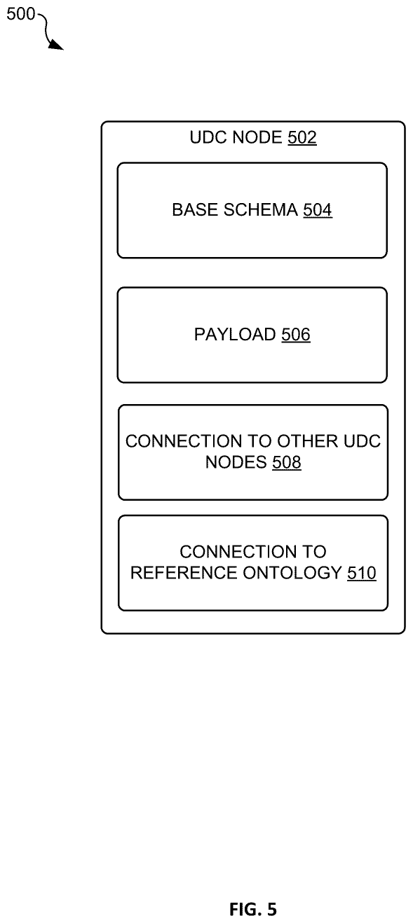

FIG. 5 illustrates a block diagram showing an example data model 500 for generating user domain concept nodes of personalized data graphs, according to at least one example. The data model 500 represents an example UDC node 502. The UDC node 502 can be modeled as having a base schema 504, a payload 506, connections to other UDC nodes 508, connections to reference ontology 510, connections to health data 512, and any other suitable element. The UDC node 502 may be included in a personalized data graph (e.g., a table that includes UDC nodes and a representation of the health data of the user). The health data may be downloaded from EHR systems, generated by the user device, obtained from a sensor of the user device or a peripheral device, and/or entered by a user of the user device.

Beginning with the base schema 504, the base schema 504 may include the following fields: a user domain concept identifier (udc_id) (e.g., a unique identifier for the UDC node, which may also correspond to a row identifier (row_id)), universal unique identifier (uuid) (e.g., an identifier that uniquely identifies the UDC node), schema (e.g., a nullable string that identifies the schema), type (e.g., an enumerated data type that distinguishes the UDC node from other types of data nodes), deleted (e.g., a “boolean” data type relating to a deleted state), creation date (e.g., information that identifies the date that the UDC node was created), modification date (e.g., information that identifies the most recent modification of the UDC node), os_version (e.g., information that identifies the version of the operating system), build (e.g., information that identifies the build of the operating system), synch anchor (e.g., information that allows tracking of which changes have not yet been pushed/pulled to/from various sync endpoints), and sync provenance (e.g., information that identifies sync origins).

In order to avoid some sync ordering issues and a large increase in the number of new sync entities, in some examples, the UDCs nodes may share a single common sync entity. In this example, the UDC type specific payload data may be serialized based on the type.

Sync anchors may allow tracking, of which changes have not yet been pushed/pulled to/from various sync endpoints. The sync anchors may be represented in the database as an integer column in the base table for a given entity. For most entities, that column may simply be the auto-incrementing rowid. In some examples, the sync anchor may increase as rows are inserted, or deleted and re-added if a “modification” is required to any row is performed.

In order to maintain device-local references between the UDC rows in various tables, the sync anchor column may be used, that is incremented when the UDC is updated. In some examples, the default conflict resolution strategy for a UDC (unique by UUID) may be “last modification wins.” This strategy may be overridden for a particular UDC type. In some examples, the UDC type identifier may be a tuple consisting of (plugin schema identifier, type enum). This may enable the integration of UDCs generation using plug ins.

Turning now to the payload 506, the payload 506 may be different depending on the UDC type. Thus, each UDC type may include custom data models, persistence (database tables), and serialization support specific to its function and purpose. For example, connections to health data 512 and connections to other UDCs nodes 508 may be implemented by the base entity. As an example, a payload for a medication example may include:
A Consumer Friendly Name (from referenced ontology)
A Nickname (user entered string)
An Icon (user selected enumerated data type)
A Status (active/inactive)

To allow for backwards/forwards compatible sync of UDCs between devices with different operating systems and reference ontology versions, UDCs may store mutable properties in a UDC property collection. This collection may be made up of generic, versioned UDC property objects. These collections of versioned properties can be merged or diffed in such a way as to keep only the latest version of a given property, while not losing properties that may be unknown (because they come from a future operating system). For example, if a future operating system version adds a property “user selected nickname” to a UDC, this property may sync back to devices running older operating system versions that do not have this property type defined and will be preserved even if other properties in the collection are modified/updated on the older device.

One such property object, can be an abstract superclass, which can be a class that needs to be instantiated by a subclass. A subclass having been defined by a special case of merging behavior, when merging properties from two collections for the same type. Addition of these subclasses can necessitate new entities to be added to the database. Subclasses that are created do not necessarily share backward compatible sync capability with pre-existing subclasses.

Turning now to the connections to other UDC nodes 508, the connections to other UDC nodes 508 may be persisted with a schema including: rowid, udc_id (foreign key referencing this UDC), connection_type (enumerated data type), target_uuid (referencing UDC UUIC of other “connected” UDC). Changes (adding or removing a connection for a UDC) may change the UDC's sync anchor, so that the UDC may sync with its current set of connections. In some examples, the target_uuid is used rather than target_udc_id. This may be helpful to support multi-device sync cases where a UDC and its connections appear on a device before all UDCs have connected to it.

Turning now to the connections to reference ontology 510, the connections to reference ontology 510 may be configured to enable efficient searching of connected reference ontology node(s) given a UDC node and to maintain connections after a restore from backup or sync to a new device. In some examples, the UDCs will persist/sync the codings for the connected ontology nodes. These codings may come from the health record the user interacted with to create the UDC, or they could be pulled from the reference ontology during a manual creation of the UDC. An example schema for these connections may include row_id, udc_id (foreign key referencing this UDC), system, code, version, and display string. In some examples, system, code, version, and display string may be nullable foreign key references to rows in a user domain concept coding strings table that exists to deduplicate storage of common coding strings. In some examples, udc_id, system, code, version may form a unique tuple. The user domain concept coding strings may include row_id, string (non-null unique string). In some examples, storing the system/codes for a UDC may avoid the need to build an index table mapping UDCs to reference ontology nodes, since these mappings can be determined by efficient run-time queries.

The default behavior for the user device can be to delete semantically equivalent concepts that are stored on the device. However, in some instances semantically equivalent concepts can persist in the user device. If, for example, semantically equivalent concepts persist, these concepts can be backed by a subclass.

Semantically equivalent concepts should be hidden from enumeration. The subclass entity that backs a semantically equivalent concept should go over a process for hiding a concept. Therefore, the concept that does not get hidden, and does not get enumerated by default is the concept with the earliest creation date. For reference, a group of semantically similar concepts can be defined as a group of concepts that share the same semantic identifier, and a canonical concept of the group is the UDC with the earliest creation date within the group. Only the canonical UDCs get enumerated by default.

Turning now to the connections to health data 512, the connections to health data 512 may be configured to enable one or more of the following: efficiently find connected health record data (e.g., samples) given UDC, efficiently find connected UDC given sample(s), handle connections to very large numbers of samples (e.g., a UDC connected with medication information), maintain or rebuild connections after restore from backup or sync to new device, function without a reference ontology, handle new medical record data represented by new UUIDs, handle change in Fast Healthcare Interoperability Resources (FHIR) identifiers (e.g., DSTU-2 to R4 conversion) for medical records, determine in-memory if a given sample is connected to a UDC (e.g., to support change detectors), serve as a concrete source without explicit mapping to related data, and support cleanup of data sourced by a UDC upon deletion.

In some examples, each UDC may persist and sync a set of “connected sample descriptions.” Any sample that matches one of these descriptions may be considered connected to the UDC. For example, a UDC that is connected to all samples with a heart rate type identifier. The connections to health data 512 may be useful for efficient bi-directional queries (e.g., samples connected to a UDC, and UDCs to which a sample is connected) if the persisted connection has a structured form in the database rather than being a serialized blob. This may allow for the direct creation of SQLite predicates that can find UDCs for a given sample and vice versa. For example, properties to represent a given sample connection description may be added as each new type of sample connection is proposed for some specific use case. The result may be a highly denormalized table, with nullable columns representing connection description specific properties.

An example schema for the connections may include rowid, udc_id (foreign key referencing this UDC), data type (nullable object type to which the connection applies), quantity (nullable health data Quantity). In some examples, certain connections may be difficult to query directly from a predicate because these connections are represented by serialized blobs in the database. In order to support querying these types of connections, the UDC node may maintain a persisted device-local UDC to sample mapping for indexed lookups in the UDC mapping entity. This mapping may be built and maintained while UDCs are generated and refreshed. The samples to be indexed may be defined in the “connected sample predicate.”

For indirect connections to samples, referencing a semantic identifier can lead to sample data on a device that can reference a concept. Data that references concepts via a semantic identifier can record concept UUIDs that create the data. If the concept gets deleted, the UDC subclass entity is responsible for cleaning up the related data for which it was the source.

Manually entered medical record samples may not have any reference coding (e.g., if they are entered for a UDC that itself is not connected to a reference ontology concept). In order to maintain the connection between the record and UDC, the manually entered record may be given some additional metadata at creation time that maps to the UDC. The connection may be maintained even if, later on the UDC and/or record begin mapping to reference ontology concepts.

In some examples, the UDC node 502 may also enable notification of user devices when UDCs are added or removed, notification of user devices when UDCs are modified, notification of user devices when new health records are connected to a UDC, and notification of user devices when new health records are added that are not connected to any UDC (e.g., so that the user can be prompted to add a UDC connection to the health record sample).

In some examples, the UDC node 502 may include relationship information that identifies, for the specific node, a relationship with respect to other nodes (e.g., UDC nodes or concept nodes). For example, such relationship information may include things such as a list member (e.g., defines that the node is a member of a list), can support (e.g., defines that the node can support), child (e.g., defines that the node is a child of a different node), parent (e.g., defines that the node is a parent of a different node), more specific (e.g., defines that the node is more specific than the other node), or less specific (e.g., defines that the node is less specific than the other node). From time to time, a user device can update a UDC. This processing operation includes logic for refreshing ontology content on a UDC, if available, and identifying and deduplicating semantically equivalent concepts. The user device can determine semantic equivalence based on a string that represents key factors that can distinguish one UDC from another UDC. For example, a UDC can include a combination of ontology coding and a UDC type identifier. In another example, a UDC can include a combination of a universal unique identifier (UUID) can a UDC type identifier. The string can be represented as an in-memory model object, which can facilitate parsing semantic identifier strings. All semantic identifiers can be represented as predicates. These predicates can be used to identify semantically equivalent concepts.

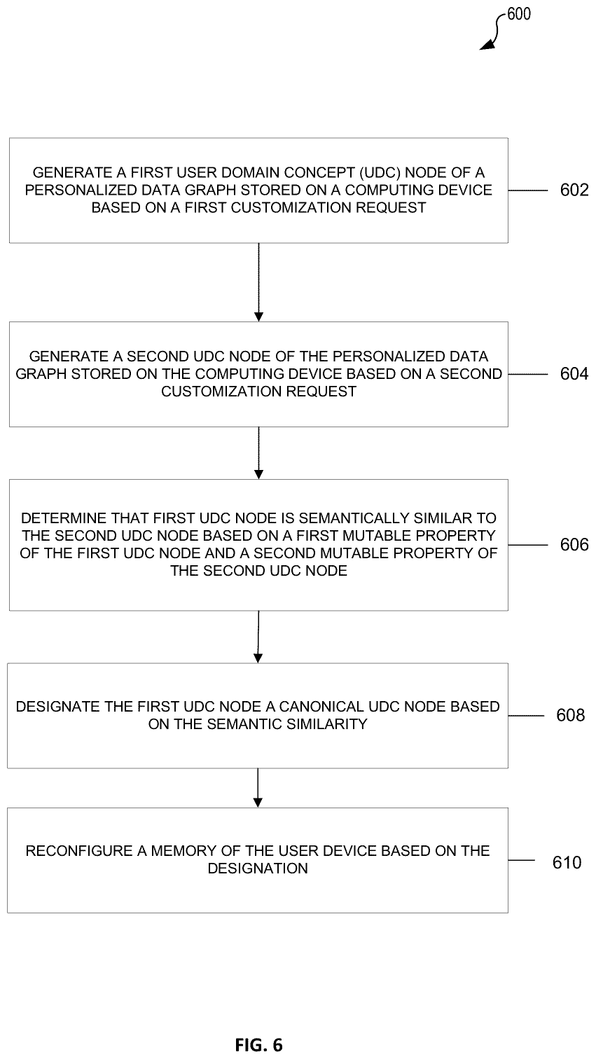

FIG. 6 is a process flow 600 for a UDC node creation, in accordance with some embodiments. At 602, a user device, (e.g., user device 104) can generate a first UDC node of a personalized data graph stored on the user device, based on a first customization request. The personalized directed graph can be associated with an embedded ontology stored on the user device. The first customization request can be based on entering a first medication identifier into the user device.

At 604, the user device can generate a second UDC node of the personalized data graph stored on the user device, based on a second customization request. The second customization request can be based on entering a second medication identifier into the user device.

At 606, the user device can determine that the first UDC node is semantically similar to the second UDC node based on a first mutable property of the first UDC node and a second mutable property of the second UDC node. The determination of semantic similarity can be based on comparing an identifying mutable property, such as a string representing an identifying property of the first UDC node, with an identifying property of the second UDC node.

At 608, the user device can designate the first UDC node as the canonical UDC node based on the determination of semantic similarity. For example, based on the determination, the user device can compare the creation dates of the first and second UDCs. The user device can further select the older UDC as the canonical UDC based on the comparison.

At 610, the user device can reconfigure its memory based on designating the first UDC node as the canonical UDC node. It should be appreciated that the memory system of the user device can be configured to continue storing the second UDC node past the designation of the first UDC node as the canonical UDC node. The second UDC node can be deleted upon request.

### Medication Interaction

A user device can provide users with the ability to determine whether any medications can have potential interactions with each other or with a lifestyle aspect of a user. The interactions can be adverse interactions that can be harmful to a user. The interactions can also be a strengthening or weakening of the potency of one or both of the interacting medications. In either case, a user should be aware of the potential for an interaction.

The user device can include an interaction detection functionality. A user can input two medications into the user device, and the device can determine whether an ingredient from one medication has an effect when combined with an ingredient of the other medication. The medications can include prescribed medication and over-the-counter medications that the user purchased without a prescription. The medications can be part of a list created by a user, derived from the user's electronic health records, inputted by a user into a medication calendar, or input in another way.

The user device can store one or more data shards that include information related to medications, including ingredients and information related to the medications. The user device can further determine a severity of the interactions (e.g., mild, moderate, severe, and contraindicated). The user device can further display the interaction, the severity of the interaction, and information related to the interaction.

A user can access the user device and input two or more medications. The user device can access an embedded ontology to retrieve information regarding the medications. The user device can apply a filter-based search to identify a respective node (e.g., information element) of the embedded ontology with information on the medications. The user device can further retrieve any child nodes of the respective medication nodes that contain additional information on medications.

Each medication can further be represented as medication product nodes in various manners, such as by product types, including packaged product, a product, a clinically normalized product or a routed dose form product. The medication product nodes can be children of the medication nodes. The user device can further traverse these child nodes of the medication nodes to detect a product type. Take, for example, the medication, Norco, which can be represented in a node as a packaged product, (Norco 10 mg-325 mg Oral Tablet made by Pfizer sold in a package of 100), as a product, (Norco 10 mg-325 mg Oral Tablet), as a clinically normalized product, (Hydrocodone bitartrate 10 mg/Acetaminophen 325 mg Oral Table), or a routed dose form product, (Hydrocodone Bitartrate/Acetaminophen Oral Tablet) in the embedded ontology.

The user device can further traverse the graph to interaction class information of the medication from interaction class nodes, which are child nodes of the medication product nodes. The user device can then determine the interaction class of the routed dose form product node. The interaction class can be based on an ingredient or a set of ingredients that behave similarly in the routed dose form product. Returning to the example above, Norco can be represented as a routed dose form product by the ingredients Hydrocodone Bitartrate and Acetaminophen. Therefore, the embedded ontology can include nodes for an interaction class of Hydrocodone Bitartrate and an interaction class node for the integration class of Acetaminophen.

The user device can determine an interaction between two medications based on detecting an interaction node, which can be a child node of the interaction class node. The user device can query the embedded ontology for interaction nodes between two interaction classes. Each interaction class node can include more than one child interaction node. For example, if a first interaction class can interact with a second interaction class and a third interaction class, the interaction class node can have a respective child interaction node for each interaction.

The user device can traverse each child node of the interaction class node to determine whether there is an interaction with an interaction class of the second medication. If the user device detects an interaction node describing an interaction with the second medication, it can display the determination for the user on the user device. Additionally, if the user device does not detect an interaction node describing an interaction with the second medication, it can also display the non-determination for the user in the user device.

The user device can further receive information regarding the user's lifestyle. This information can include diet, exercise routine, biometric data, health state, and other lifestyle related information. For example, the user can input the lifestyle related information into the user device, such as diet or exercise routines. In other instances, the user can allow the user device to receive biometric data collected by a sensor. The user device can use the collected data to make assumptions about the user's lifestyle.

The determination of whether the first medication interaction with a lifestyle aspect of the user is similar to the determination of whether the first medication interacts with the second medication. In this instance, the user device proceeds the same way as identifying the interaction class node of the first medication. Upon identifying the interaction class node, the user device can search for interaction nodes that describe and an interaction between the interaction class and a lifestyle aspect of the user.

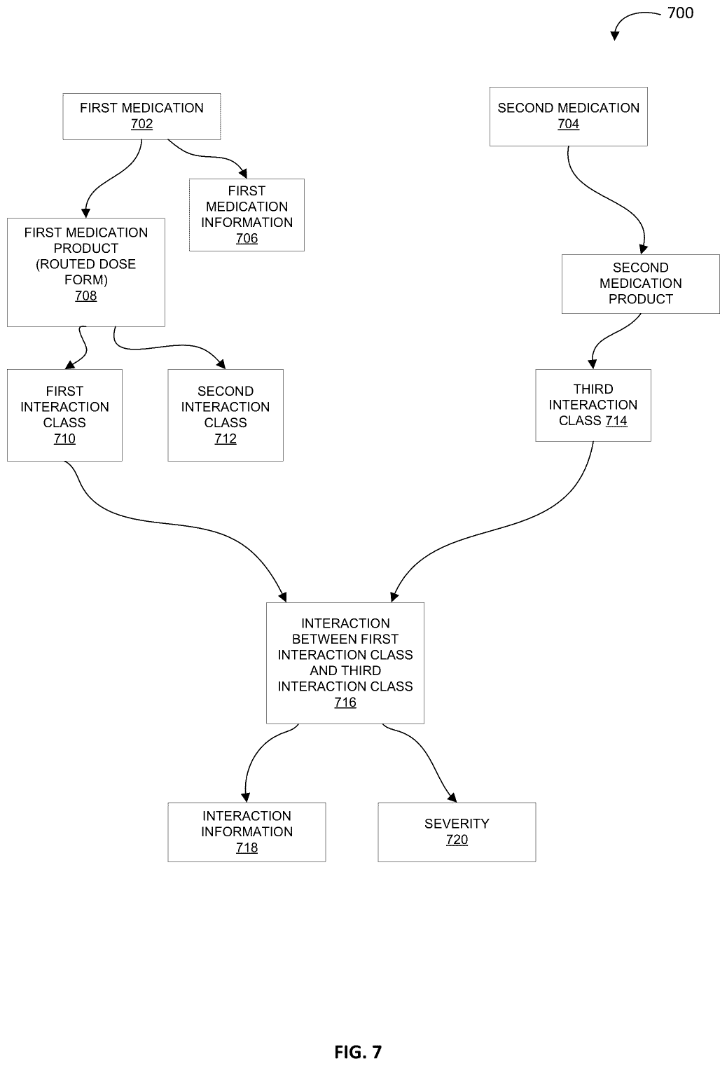

FIG. 7 is an illustration 700 of a medication interaction detection process, in accordance with some embodiments. A user can enter a name of a first medication and a second medication into the user device (e.g., user device 104). The user device can detect both the first medication and the second medication and discover corresponding nodes in an embedded ontology stored on the user device. The user device can search for the two medications by a brand name, generic name, pharmaceutical name, or other name supported by the embedded ontology. It should be appreciated that the user device follows a same path for the first medication and the second medication. For the sake of brevity, the path followed by the user device in relation to the first medication is described.

The user device can detect the node for the first medication node 702 in the embedded ontology. The user device can further traverse each child node of the first medication node 702 and discover a first medication information node 706 and a first medication product node 708. The first medication information node 706 can include general information, including the type of medication, common side effects, common allergic reactions, and a geographic region the medication is available.

The first medication product node 708 can include packaged product, a product, a clinically normalized product or a routed dose form product node. For purposes of illustration, only the routed dose form product node is shown. Based on the first medication product node 708, the user device can identify one or more interaction class nodes associated with the first medication. The interaction class nodes can describe the ingredients of the first medication. For example, Norco in a routed dose form can include Hydrocodone Bitartrate and Acetaminophen. Using this example, the daemon would recognize two interaction class nodes, one for Hydrocodone Bitartrate and another for Acetaminophen.

As illustrated, the user device can traverse the embedded ontology and find a first interaction class node 710 and a second interaction class node 712, wherein each of the interaction class nodes is a child node of the first medication product node 708.

The user device can continue to traverse the embedded ontology and detect one or more interaction nodes. The interaction nodes can be nodes that describe an interaction between two or more interaction classes. Each of the first interaction class and the second interaction class can interact with multiple different interaction classes. For the purposes of this diagram, the user device traverses the embedded ontology for an interaction node that includes an interaction for the first interaction class and an interaction class associated with the second medication.

As illustrated, the user device can locate a third interaction node 714 describing an interaction between the first interaction class and a third interaction class of the second medication. In response to detecting an interaction between the first medication and the second medication, the user device can continue to traverse the embedded ontology for the child nodes of the interaction node 716. The user device can find an interaction information node 718 that includes information or access to information regarding the interaction. The user device can also find the severity node 720, which describes the severity of the interaction. The user device can further display the information and the severity for the user.

In response to determining that an interaction can occur between the first medication and the second medication, the user device can display the interaction, any interaction related information, and the severity of the interaction.

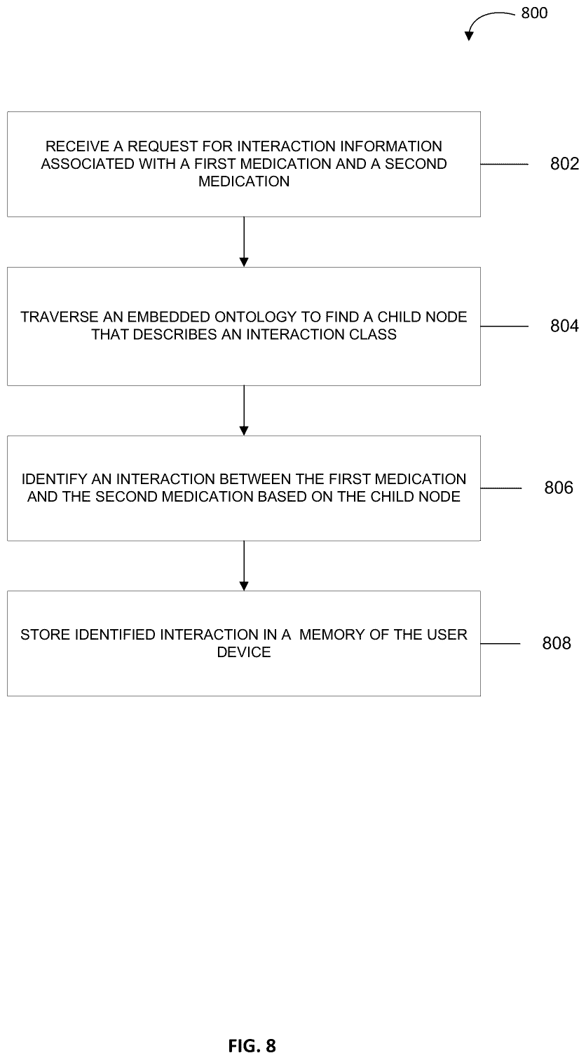

FIG. 8 is a process flow 800 for a medication interaction detection process, in accordance with some embodiments. At 802, a user device (e.g., user device 104) can receive a request for interaction information associated with a first medication and a second medication. The request may be received as a user input received at the user device.

At 804, the user device can traverse an embedded ontology to find a child node that describes an interaction class. For example, the user device can access an embedded ontology stored on the user device and detect a node for the first medication. As described with reference to FIG. 7, the process the user device follows with respect to the first medication is described, with the understanding that the user device follows the same process for the second medication.

The user device can traverse the embedded ontology to find a child node of the first medication node that describes a product type of the first medication, including packaged product, a product, a clinically normalized product, or a routed dose form product.

The user device can traverse the embedded ontology to find each child node of the product type node that describes an interaction class of the first medication. An interaction class can be, for example, an ingredient of the first medication.

At 806, the user device can traverse the embedded ontology to find each child node of the interaction class nodes that described an interaction with an interaction class of the second medication. The interaction node can further lead to an interaction information node, if any, and a severity node, if any, that provides information on the interaction and a severity of the interaction.

At 808, the user device can store the interaction in memory, e.g., cache memory on the user device. In addition, the user device can display the interaction, any interaction information, and any severity of the interaction. The interaction information and the severity information can be described, for example, in child nodes of the interaction node. In some instances, detecting the interaction node or the severity node can trigger the user device to cause a request for an ontology shard from a global ontology (e.g., global ontology 110) to be retrieved from a server 108. The ontology shard can be integrated with an embedded ontology (e.g., embedded ontology 106) stored on the user device (e.g., user device 104).

As suggested above, the user device can also determine an interaction between a medication and a lifestyle aspect of the user. For example, the user can input his or her dietary choices into the user device. In this instance, a node in a directed graph can be created for the lifestyle aspect of the user.

Embodiments herein describe a computer-implemented methodology for medication interaction detection that can be executed locally on a user device to preserve the confidential data of a user. In particular, the user device can receive and store medication information locally in the user device's memory. Furthermore, a processor of the user device can use the locally stored information to identify potential interactions between two or more medications. This information can be displayed to the user on the user device's display. Therefore, the herein described embodiments do not require a user device to transmit personally identifiable information to an external device, such as a server, have the server process the information to detect any potential interactions, and then transmit the analysis back to the user device. Furthermore, the herein described embodiments reduce the complexity of the traditional process, as any transmission to an external device would require an encryption on the transmitting end and a decryption on the receiving end. As the herein described methodology is performed locally, the process does not require the added complexity of encryption and decryption of transmissions and receptions.

### Medication Calendaring

A user device can include an electronic medication calendaring system that allows users to organize and manage their medications. The user device can receive a medical identifier for one or more medications and prescribed frequency for taking the one or more medications. The user device can further determine when a user needs to take a medication at a future date based on the medication and the frequency that the patient is supposed to take the medication.

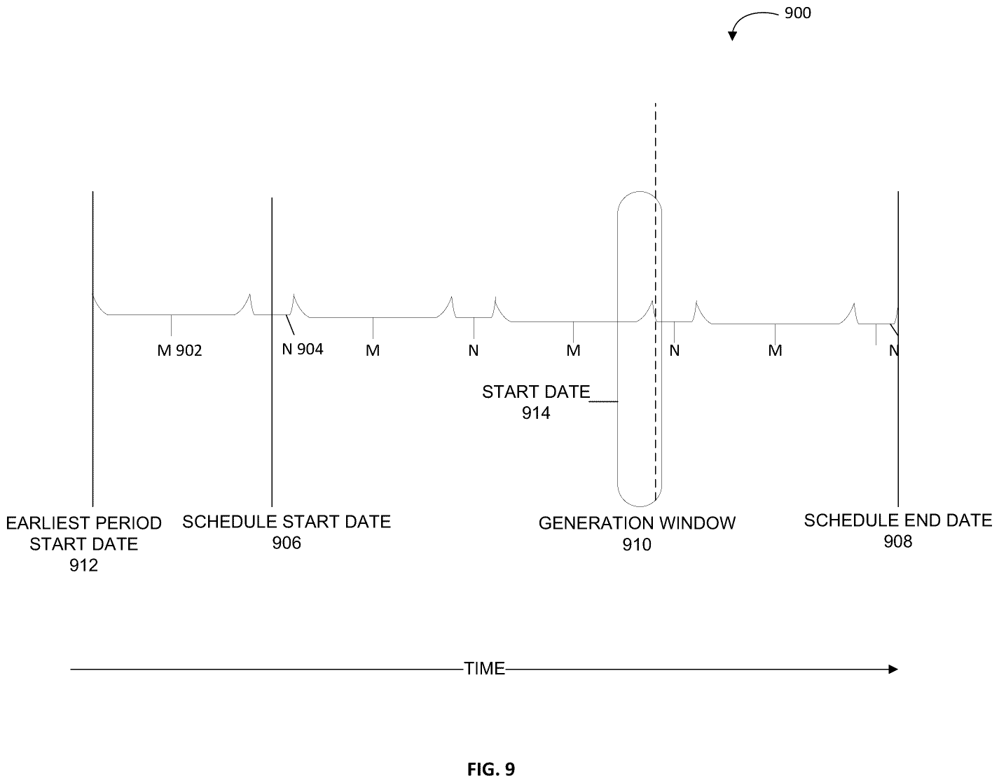

FIG. 9 is an illustration 900 of a medication calendaring system, in accordance with some embodiments. A user can enter a first interval of days 902 and a second interval of days 904. The first interval of days 902 can be for “M” days” and the second interval of days 904 can be for “N” number of days and run consecutively to the first interval of days 902 into the user device. In some embodiments, the first interval of days 902 can be days in which the user is directed to take the medication. The second interval of days 904 can be days that the user is directed to refrain from taking the medication. In some other embodiments, the first interval of days 902 can be days in which the user is directed to take a first medication. The second interval of days 904 can be days that the user is directed to take a second medication.

The user can further enter a schedule start date 906 into the user device, the date being the date that the user is to begin taking the medication. The schedule start date is not necessarily the date that the user began inputting the information into the user device. For example, the user can begin inputting information for medication calendaring before, during, or after the schedule start date 906. The user can further enter a schedule end date 908 describing a date to cease calendaring the medication.

The user device can determine a number of days for a medication period, which can be the sum of the first interval of days 902 and the second interval of days 904 (e.g., M+N=medication period). The user device can further populate a timeline with the repeating periods of the first interval of days and the second interval of days.

The user device can further create a generation window 910, where the start date of the generation window 910 is a date after the schedule start date 906. The generation window can be a slidable window that can be manipulated by a user, including a number of days included in the generation window. The slidable window can be the dates for which the user wants an automatic medication calendaring.

The user device can then detect an earliest period start date 912, which can be a first medication period in time that includes the schedule start date 906. The user device can then determine whether the period includes the start date of the generation window 914. As illustrated, the first medication period includes the earliest period start date 912 but does not include the start date of the generation window 910. The user device continues to verify each consecutive period to determine whether the period includes the start date of the generation window 914.

In response to determining that the medication period includes the start date of the generation window 914, the user device determines that this medication period is the current period.

In response to determining that a period is the current period, the user device can populate days on a calendar that correspond to days of the generation window with an indication of whether or not to take a medication (e.g., first medication or second medication). Using the example from above in which the user takes medication for the first interval of days 902 and refrains from taking medication the second interval of days 904, the user device can populate the days that correspond to the first interval of days 902 with notifications to take the medication, whereas the user device can leave the days associated with the second interval of days 904 blank as the user is to refrain from taking the medication.

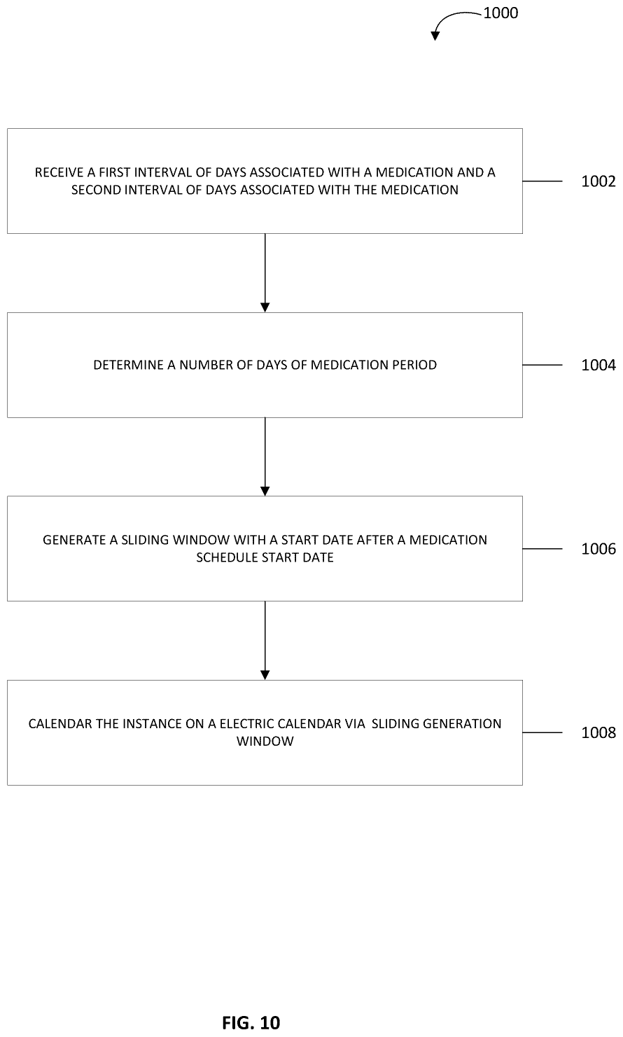

FIG. 10 is a process flow 1000 for medication calendaring, in accordance with some embodiments. At 1002, a user device (e.g., user device 104), can receive a first interval of days and a second interval of days. Each interval of days can be associated with a medication. In some instances, the medication can be associated with a medication identifier stored on an embedded ontology (e.g., embedded ontology 106). In response to the user inputting the medication, the user can retrieve the medication identifier from the embedded ontology. The user device can further retrieve information from the embedded ontology related to the medication based on the medication identifier. In even further embodiments, the user device can be triggered to request an ontology shard from a global ontology (e.g., global ontology 110).

In some embodiments, the first interval of days can be associated with a first medication and the second interval of days can be associated with refraining from taking the medication. In other embodiments, the first interval of days can be associated with taking a first medication, and the second interval of days can be associated with taking a second medication.

At 1004, the user device can determine a number of days of a medication period. In some embodiments, the user device calculates a sum of the number of days in the first interval of days and the number of days in the second interval of days.

At 1006, the user device can generate a sliding window with a start date after a medication schedule start date. The sliding window can be a window for populating a medication calendar with indications of whether to or not to take a medication. In some embodiments, the user device can further determine whether the start date of the sliding window fall after the medication schedule start date.

In some instances, a user has more than one device, such as a user device (e.g., user device 104), and another device, such as a laptop. The user device can further cause a synchronization of a calendar stored on the user device with a calendar stored on the other device. For example, the user device can open a secure channel with the other device and exchange information to synchronize the calendars.

As described above, the period includes a first interval of days and a second interval of days. Therefore, the user device can further determine which days within the window fall within the first interval of days and which days fall within the second interval of days.

At 1008, the user device can calendar an instance of the medication within the generation window. The calendaring can be based on whether a data falls within the first interval of days or the second interval of days.

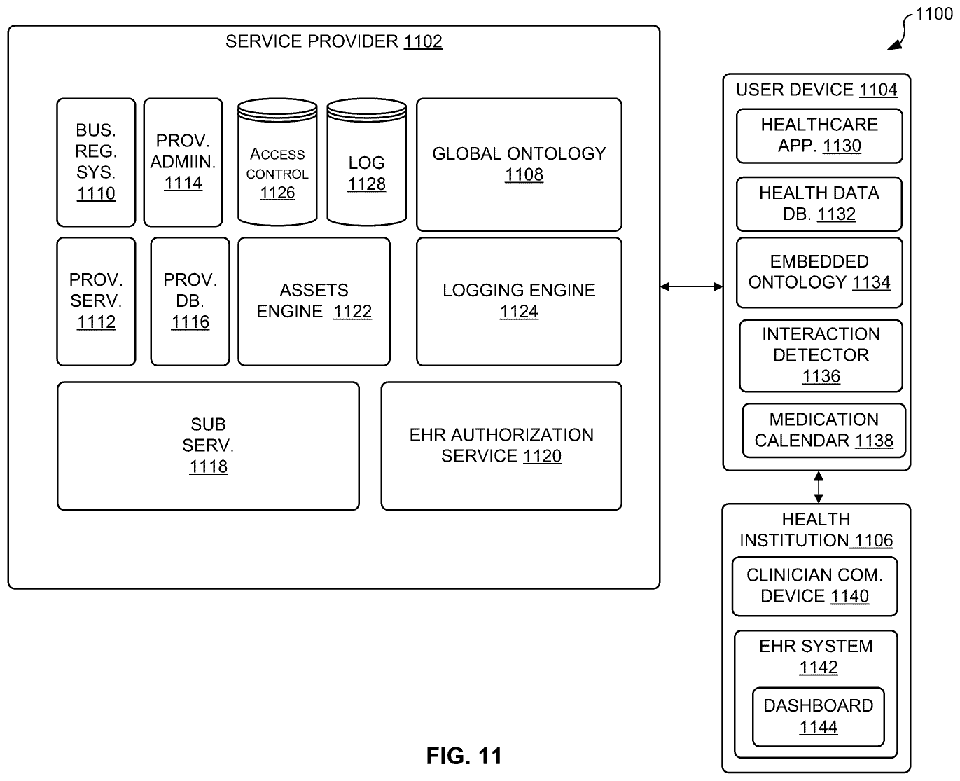

Embodiments herein describe a computer-implemented methodology for locally generating and displaying a medication calendar. In particular, the user device can receive medication information for generating a computer-generated visualization of a medication schedule. The user device can further generate notifications for the user with respect to upcoming scheduled medications. Conventional calendaring systems rely on cloud-based storage for storing a personal calendar. Therefore, even if the data is encrypted the user's data is stored on an external storage device and exposed to potential malfeasance. The herein described embodiments permit a user device to receive medication calendaring information from the user via a user interface. A user device can use the information to determine a medication calendar for the user. The user device can further locally generate and control instructions necessary to display the medication calendar on the user interface displayed on the user device. Furthermore, as indicated above, the herein described embodiments reduce the complexity of the traditional process, as any transmission to an external device would require an encryption on the transmitting end and a decryption on the receiving end. As the herein described methodology is performed locally, the process does not require the added complexity of encryption and decryption of transmissions and receptions. FIG. 11 is an illustration of system 1100 for executing a health application, in accordance with some embodiments. The system 1100 is in communication with a service provider 1102, (e.g., a publisher, such as Apple, of the health application), the user device 1104 (e.g., user device 104), and the health institution 1106. The service provider 1102 can be, for example, an entity responsible for managing a global ontology (e.g., global ontology 110), including updating an embedded ontology (e.g., embedded ontology 106), on a user device (e.g., user device 104). As appropriate and as illustrated by the arrows, the elements of the system 1100 may be communicatively coupled via one or more suitable communication networks. For example, the service provider 1102, the user device 1104, and the health institution 1106 may be configured to communicate with each other via one or more networks, as described herein and is known in the art.

The service provider 1102 includes one or more server computers, which may be virtual, and is configured to manage application executing on the user device 1104. In particular, different elements of the service provider 1102 manage different aspects of sharding, including authorization of user devices 1104, data retrieval, and the like.

The service provider 1102 includes a global ontology 1108 (e.g., global ontology 110), a business registration system 1110, a provider service 1112, a provider administrator 1114, a provider database 1116, a subscription service 1118, an electronic health records (EHR) authorization service 1120, an assets engine 1122, a logging engine 1124, an access control storage 1126, and a log 1128.

The user device 1104 may include a health application 1130, the health data database 1132, an embedded ontology 1134 (embedded ontology 106), an interaction detector 1136, and a medication calendar 1138. Generally, the user devices 1104 may be associated with and operated by different users. Functionally, the health application 1130 may enable the user devices 1104 to communicate with the service provider 1102, communicate with the EHR systems 1142 of the health institutions 1106.

The health institution 1106 can include a system of computers including computing devices, such as smartphones, laptops, and servers. The health institution can include a clinician computing device 1140, such as a smartphone or an office computer. The health institution can further include an electronic health record (EHR) system 1142, including a dashboard 1144.

Returning to the service provider 1102, the global ontology 1108 can be a representation of categories and relationships between health concepts in an overall health domain. The global ontology 1108 can be stored on a server and be arranged as a data structure such as a directed graph, in which concepts are described as graph nodes and related nodes are connected via edges. Each node can include information related to a health concept. For example, a node can include a uniform resource location (URL) for a site containing information related to a concept.

The global ontology 1108 can be partitioned into a collection of shards, where each shard is a partition of health-related data related to a core concept in the health domain. Each shard can include a stable identifier that uniquely identifies the shard. Each shard further includes a set of nodes that relate to a core health concept. The nodes of each shard can further be connected by edges that describe the relationships between the nodes. Each shard can include multiple shard instances that include the same nodes and edges but are formatted differently to be compatible with different operating systems or versions of an operating system.

The service provider 1102 can, from time to time, update one or more shards of the global ontology 1108, to update information in a node or a connection between two or more nodes. The service provider 1102 can further transmit an updated shard to a user device 1104. For example, in response to a request for information relating to a shard. In other instances, the service provider 1102 can transmit an updated shard to the user device 1104 based on a previous request for information.

The business registration system 1110 may be any suitable collection of hardware and software components configured to collect, store, update, and otherwise manage business locations including those of partner institutions. For example, the business registration system 1110 may include a business database and a subscription service to enable subscription of the service provider 1102. When a partner is subscribed and active, the user devices 1104 may be allowed to share health data with the health institution 1106 and connect to and download health record data from an electronic health record (EHR) system 1142. The partner can be, for example, a third-party medical records service provider.

As part of subscribing and managing subscriptions, the subscription service 1118 may include functionality to collect, store, update, and otherwise manage business locations. In some examples, the subscription service 1118 provides one or more user interfaces by which authorized users of the health institution 1106 may input information about their location. This information may include geographic information (e.g., a physical address and pin on a map), image information (e.g., logos), contact information (e.g., business, legal, technical), and any other information relating to a business. The subscription service 1118 may also be configured to create and/or update record entries in the business registration system 1110 based on information received. For example, an authorized user associated with the health institution 1106 may share business information with the subscription service 1118. Once this information has been shared and validated, the business information may be published for public consumption (e.g., indexed for searching, made available on a map platform, shared directly with users).

The business registration system 1110 may maintain the collected business information and any relationships between entities represented by the business information. In some examples, the business registration system 1110 may be used to register other business entities (not just health institutions 1106). Records for the health institutions 1106 may be maintained in both the business registration system 1110 and a provider database 1116 maintained by the service provider 1102. In some examples, the business registration system 1110 shares business information with the service provider 1102 in any suitable manner.

Turning now to the provider service 1112, generally, the provider service 1112 may validate the EHR systems 1142, maintain information about the health institutions 1106 and associated EHR systems 1142, enable searching of the health institutions 1106 associated with the EHR systems 1142, and manage access of the user devices 1104 to the EHR systems 1142. The service provider 1102 can implement one or more of the described elements in a server as part of the “cloud.” The provider service 1112 may also maintain information about the health institutions 1106 and associated EHR systems 1142 in the provider database 1116, enable searching of the health institutions 1106 associated with the EHR systems 1142, and manage access of the user devices 1104 to the EHR systems 1142. In some examples, the user device 1104 can send requests to search for the health institutions 1106 to the provider service 1112. The provider service 1112 processes these requests and returns results. In some examples, as part of establishing a connection with one of the EHR systems 1142, the user device 1104 can check in with the provider service 1112 to determine whether any configuration information associated with the EHR system 1142 has changed. The configuration information, which may be stored in the provider database 1116, may include API information, provider identifiers, status indicator information, and any other suitable information relating to the configuration of the EHR system 1142 and/or other entities associated with the EHR system 1142. The provider administrator 1114 can generally be used by an administrator or other authorized user to manage aspects of the provider service 1112.

The service provider 1102 may include an assets engine 1122, a logging engine 1124, access control storage 1126, and a log 1128. The assets engine 1122 can be configured to manage the assets of the service provider 1102. This may include removing or otherwise deleting old or stale data in storage. In some examples, such deletion may be part of a garbage collection routine or may be requested or otherwise instructed by the user device 1104. For example, the user device 1104 may change the way the health data is stored, and the assets engine 1122 may make the adjustments in storage.

The clinician computing device 1140 may be operated by a clinician or other authorized user to access the health data of the user. The dashboard 1144, which may be presented by the clinician computing device 1140, may be an application such as web application accessible via a web browser of the clinician computing device 1140 or any other suitable application accessible by the clinician computing device 1140. In some examples, the dashboard 1144 may be hosted by the EHR system 1142 and may be the typical dashboard 1144 used by the clinician to access electronic health records stored by the EHR system 1142. To enable the dashboard 1144 to view the health data from the service provider 1102, as described herein, the dashboard 1144 may be slightly modified to include a link, icon, or other graphical element to enable the clinician to see the health data loaded into the EHR using the techniques described herein. In some examples, EHR system 1142 communicates with the health application 1130 to authenticate the user of the user device 1104. Such communications may occur using an existing Health Level Seven International Standard (HL7) such as Fast Health Interoperability Resource (FHIR) standard describing data elements, data formats, and APIs for exchanging electronic health records. In some examples, the open source implementation of the FHIR standard, referred to as SMART on FHIR (Substitute Medical Applications, Reusable Technologies on FHIR), may be appropriate.

The user device 1104 may include the health application 1130 and the health data database 1132. As described herein, the health application 1130 may be used to perform the techniques relating to generating UDC nodes, storing health data using such nodes, and the like. The health data database 1132 may be used to store the health data that is shared, as described herein, and any other health record data.

The user device 1104 can include an embedded ontology 1134, which includes a subset of ontology shards received from the global ontology 1108. The user device 1104 further includes an interaction detector 1136, which can be a backend process of the health application 1130 that can receive inputs, such as medication and/or lifestyle aspects from a user and determine whether the medication interacts with each other or a lifestyle aspect. The interaction detector is further operable to retrieve interaction data from the embedded ontology 1134. The user device 1104 can further include a medication calendar 1138 for generating a medication calendar for a user.

The logging engine 1124 may be used to log transaction information related to requests for healthcare data received from the clinician dashboard 1144 and/or the clinician computing device 1140, as described in further detail herein. In some examples, the logging engine 1124 may also include encryption and/or decryption algorithms for performing cryptographic functions, as described herein. The logging engine 1124 may store transaction information in the health data database 1132, as introduced herein.

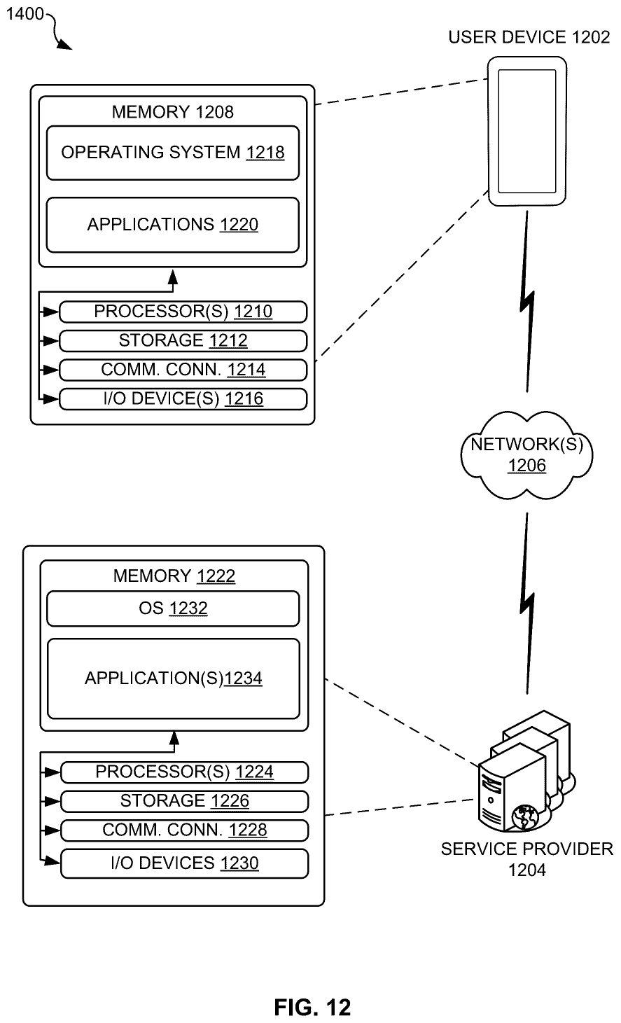

FIG. 12 is an illustration of a computing architecture 1200, in accordance with some embodiments. In some examples, the example architecture 1200 may further be configured to enable a user device 1202 (e.g., user device 104) and service provider computer 1204 to share information. The service provider computer 1204 is an example of the service provider 1102 and the EHR system 1242. The user device 1202 is an example of the user device 104. In some examples, the devices may be connected via one or more networks 1206 (e.g., via Bluetooth, Wi-Fi, or the Internet). In some examples, the service provider computer 1204 may be configured to implement at least some of the techniques described herein with reference to the user device 1202 and vice versa.

In some examples, the networks 1206 may include any one or a combination of many different types of networks, such as cable networks, the Internet, wireless networks, cellular networks, satellite networks, other private and/or public networks, or any combination thereof. While the illustrated example represents the user device 1202 accessing the service provider computer 1204 via the networks 1206, the described techniques may equally apply in instances where the user device 1202 interacts with the service provider computer 1204 over a landline phone, via a kiosk, or in any other manner. It is also noted that the described techniques may apply in other client/server arrangements (e.g., set-top boxes), as well as in non-client/server arrangements (e.g., locally stored applications, peer-to-peer configurations).

As noted above, the user device 1202 may be any type of user device such as, but not limited to, a mobile phone, a smartphone, a personal digital assistant (PDA), a laptop computer, a desktop computer, a thin-client device, a tablet computer, a wearable device such as a smartwatch, or the like. In some examples, the user device 1202 may be in communication with the service provider computer 1204 via the network 1206 or via other network connections.

In one illustrative configuration, the user device 1202 may include at least one memory 1208 and one or more processing units (or processor(s)) 1210. The processor(s) 1210 may be implemented as appropriate in hardware, computer-executable instructions, firmware, or combinations thereof. Computer-executable instruction or firmware implementations of the processor(s) 1210 may include computer-executable or machine-executable instructions written in any suitable programming language to perform the various functions described. The user device 1202 may also include geo-location devices (e.g., a global positioning system (GPS) device or the like) for providing and/or recording geographic location information associated with the user device 1202.

The memory 1208 may store program instructions that are loadable and executable on the processor(s) 1210, as well as data generated during the execution of these programs. Depending on the configuration and type of the user device 1202, the memory 1208 may be volatile (such as random access memory (RAM)) and/or non-volatile (such as read-only memory (ROM), flash memory). The user device 1202 may also include additional removable storage and/or non-removable storage 1212 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 1208 may include multiple different types of memory, such as static random access memory (SRAM), dynamic random access memory (DRAM), or ROM. While the volatile memory described herein may be referred to as RAM, any volatile memory that would not maintain data stored therein once unplugged from a host and/or power would be appropriate.

The memory 1208 and the additional storage 1212, both removable and non-removable, are all examples of non-transitory computer-readable storage media. For example, non-transitory computer-readable storage media may include volatile, or non-volatile, removable or non-removable media implemented in any method or technology for storage of information such as computer-readable instructions, data structures, program modules, or other data. The memory 1208 and the additional storage 1212 are both examples of non-transitory computer-storage media. Additional types of computer-storage media that may be present in the user device 1202 may include, but are not limited to, phase-change RAM (PRAM), SRAM, DRAM, RAM, ROM, Electrically Erasable Programmable Read-Only Memory (EEPROM), flash memory or other memory technology, compact disc read-only memory (CD-ROM), digital video disc (DVD) or other optical storage, magnetic cassettes, magnetic tape, magnetic disk storage or other magnetic storage devices, or any other medium that can be used to store the desired information and that can be accessed by the computing device. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media. The memory 1208 and/or the additional storage 1212 may store the health data database 1132 of FIG. 11 and/or the embedded ontology 1134 of FIG. 11.

The user device 1202 may also contain communications connection(s) 1214 that allow the user device 1202 to communicate with a data store, another user device or server, user terminals, and/or other devices via the network 1206. The user device 1202 may also include I/O device(s) 1216, such as a keyboard, a mouse, a pen, a voice input device, a touch screen input device, a display, speakers, and a printer.

Turning to the contents of the memory 1208 in more detail, the memory 1208 may include an operating system 1218 and/or one or more application programs or services for implementing the features disclosed herein such as applications 1220 (e.g., health application 1130, interaction detector 1136, medication calendar 1138). In some examples, the applications 1220 may include a health application (e.g., the health application 1130) to perform similar techniques as described with reference to the user device 1202. Similarly, at least some techniques described with reference to the service provider computer 1204 may be performed by the user device 1202.

The service provider computer 1204 may also be any type of computing device such as, but not limited to, a collection of virtual or “cloud” computing resources, a remote server, a mobile phone, a smartphone, a PDA, a laptop computer, a desktop computer, a thin-client device, a tablet computer, a wearable device, a server computer, or a virtual machine instance. In some examples, the service provider computer 1204 may be in communication with the user device 1202 via the network 1206 or via other network connections.

In one illustrative configuration, the service provider computer 1204 may include at least one memory 1222 and one or more processing units (or processor(s)) 1224. The processor(s) 1224 may be implemented as appropriate in hardware, computer-executable instructions, firmware, or combinations thereof. Computer-executable instruction or firmware implementations of the processor(s) 1224 may include computer-executable or machine-executable instructions written in any suitable programming language to perform the various functions described.

The memory 1222 may store program instructions that are loadable and executable on the processor(s) 1224, as well as data generated during the execution of these programs. Depending on the configuration and type of service provider computer 1204, the memory 1222 may be volatile (such as RAM) and/or non-volatile (such as ROM and flash memory). The service provider computer 1204 may also include additional removable storage and/or non-removable storage 1226 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 1222 may include multiple different types of memory, such as SRAM, DRAM, or ROM. While the volatile memory described herein may be referred to as RAM, any volatile memory that would not maintain data stored therein, once unplugged from a host and/or power, would be appropriate. The memory 1222 and the additional storage 1226, both removable and non-removable, are both additional examples of non-transitory computer-readable storage media.

The service provider computer 1204 may also contain communications connection(s) 1228 that allow the service provider computer 1204 to communicate with a data store, another computing device or server, user terminals, and/or other devices via the network 1206. The service provider computer 1204 may also include a communications connection 1228 and I/O device(s) 1230, such as a keyboard, a mouse, a pen, a voice input device, a touch input device, a display, speakers, and a printer.

Turning to the contents of the memory 1222 in more detail, the memory 1222 may include an operating system 1232 and/or one or more application programs 1234 or services for implementing the features disclosed herein including a provider service 1112, the provider admin 1114, the subscription service 1118, business registration system 1110, the logging engine 1124, the access control storage 1126, health data database 1132, and/or the dashboard 1144.

### Examples

In the following sections, further example embodiments are provided.

Example 1 includes a method, the method includes a user device receiving an instruction to fetch data having a first search criterion; retrieving, by the user device, a first shard including data matching the first search criterion, wherein, the first shard, by the user device, is from an embedded ontology stored on the user device in accordance with a determination that the data matching the first search criterion is in the embedded ontology; or the first shard, by the user device, is from a global ontology on a remote server in accordance with a determination that data matching the first search criterion is not in the embedded ontology; and displaying, by the user device, a portion of the first shard to the user.

Example 2 includes the method of example 1, wherein the instruction persists past the transmission of the first shard, and the method further includes transmitting a second shard based on an update of the first shard, the second shard being an updated first shard.

Example 3 includes the method of any of examples 1 and 2, wherein the method further includes receiving a second instruction to fetch different data having the first search criterion; and updating the first shard with information from the global ontology when the different data is distinct from the data.

Example 4 includes the method of any of examples 1-3, wherein the first shard is stored in a first file format and a second file format, and the method further includes selecting the first file format of the first shard based on compatibility with the user device.

Example 5 includes the method of any of examples 1-4, wherein the method further includes receiving an indication of a geographical region associated with the user device; and transmitting the first shard to the user device from the global ontology based on the geographical region.

Example 6 includes the method of any of examples 1-5, wherein the first search criterion includes a medication interaction or a medication.

Example 7 includes the method of any of examples 1-6, wherein the global ontology is a medical ontology.

Example 8 includes the method of any of examples 1-7, wherein the method further includes traversing the embedded ontology in response to receiving the instruction to fetch data having a first search criterion; determining that the embedded ontology does not include data having the first search criterion; and transmitting a request to the global ontology for data having the first search criterion, based at least in part on the determination.

Example 9 includes the method of example 8, wherein the first shard is integrated into an embedded ontology of the user device.

Example 10 includes the method of example 8, wherein the instruction includes a location of the user device, and wherein receiving the first shard is based on the location.

Example 11 includes a user device including means to perform one or more elements of a method described in or related to examples 1-10.

Example 12 includes a non-transitory computer-readable media including instructions to cause a user device, upon execution of the instructions by one or more processors of the user device, to perform one or more elements of a method described in or related to any of examples 1-10.

Example 13 includes a method, the method includes a user device generating a first UDC node of a personalized data graph stored on the user device, based on a first customization request; generating, by the user device, a second UDC node of the personalized data graph based on a second customization request; determining, by the user device, that the first UDC node is semantically similar to the second UDC node based on a first mutable property of the first UDC node and a second mutable property of the second UDC node; designating, by the user device, the first UDC node as a canonical UDC node based on the determination of semantic similarity; and reconfiguring, by the user device, a memory of the user device based the designation.

Example 14 includes the method of example 13, wherein the method further includes persisting storage of the second UDC node in the user device past the designation of the first UDC node as the canonical UDC node.

Example 15 includes the method of any of examples 13 and 14, wherein the first customization request is storing a first medication identifier in the user device.

Example 16 includes the method of any of examples 13-15, wherein designating the first UDC node as the canonical UDC node includes comparing a creation date of the first UDC node and a creation date of the second UDC node; and selecting the first UDC node based on the comparison.

Example 17 includes the method of any of examples 13-16, wherein the method further includes receiving a request to delete the first UDC node; and deleting the first UDC node and the second UDC node based on the request to delete the first UDC node, wherein a first data associated with the first UDC node and a second data associated with the second UDC node persist past the deletion.

Example 18 includes the method of any of examples 13-17, wherein the first UDC node includes a base schema, a connection to a sample, a connection to the second UDC node, and a payload.

Example 19 includes the method of any of examples 13-18, wherein the first mutable property includes a string representing an identifying parameter of the first UDC node.

Example 20 includes the method of any of examples 13-19, wherein the first UDC node includes medication interaction information.

Example 21 includes a user device including means to perform one or more elements of a method described in or related to examples 13-20.

Example 22 includes a non-transitory computer-readable media including instructions to cause a user device, upon execution of the instructions by one or more processors of the user device, to perform one or more elements of a method described in or related to any of examples 13-20.

Example 23 includes a method for detecting drug interactions on a user device, the method includes the a user device receiving a request for interaction information associated with a first medication; traversing an embedded ontology to find a child node that describes an interaction class; identifying, by the user device, an interaction between the first medication and a second medication based on the child node; and storing, by the user device, the identified interaction in memory of the user device.

Example 24 includes the method of example 23, wherein the method further includes identifying an ingredient type of a first ingredient by performing a lookup on an embedded ontology based on a medication identifier; determining a routed dose form product based on the ingredient type; and determining an ingredient class based in the routed dose form product.

Example 25 includes the method of any of examples 23 and 24, wherein the embedded ontology includes a node describing a severity of the interaction between the first ingredient and a second ingredient.

Example 26 includes the method of any of examples 23-25, wherein the method further includes retrieving the identified interaction from the memory; and presenting the identified interaction between the first medication and the second medication on the user device.

Example 27 includes the method of any of examples 23-26, wherein the embedded ontology includes a node representing a lifestyle parameter.

Example 28 includes the method of any of examples 23-27, wherein the method further includes requesting a shard based on the identified interaction; and integrating the shard with an ontology framework stored on the user device.

Example 29 includes the method of any of examples 23-28, wherein the method further includes generating a user domain concept (UDC) node based on the first medication, and wherein the UDC node provides a link between the first medication and an ontology framework stored on the user device.

Example 30 includes a user device including means to perform one or more elements of a method described in or related to examples 23-29.

Example 31 includes a non-transitory computer-readable media including instructions to cause a user device, upon execution of the instructions by one or more processors of the user device, to perform one or more elements of a method described in or related to any of examples 23-29.

Example 32 includes a method, the method including a user device receiving a first interval of days associated with a medication identifier and a second interval of days associated with the medication identifier, wherein the second interval of days sequentially follows the first interval of days; determining, by the user device, a number of days of a medication period, the medication period including the first interval of days followed by the second interval of days; generating, by the user device, a second instance of the medication identifier; and calendaring, by the user device, the second instance on an electronic calendar via a sliding generation window, the sliding generation window including a consecutive set of days of the days of the medication period

Example 33 includes the method of example 32, wherein the first interval of days is designated for taking a first medication, and wherein the second interval of days is designated for refraining from taking the first medication.

Example 34 includes the method of any of examples 32 and 33, wherein calendaring the second instance includes creating the sliding generation window after an initial medication date; determining whether a selected day of the sliding generation window falls within the first interval of days or the second interval of days; and calendaring the second instance based on the determination of whether the selected day falls within the first interval of days or the second interval of days.

Example 35 includes the method of any of examples 32-34, wherein the electronic calendar is synchronized between the user device and another device, and wherein the method further includes displaying a notification on either the first device or second device based on a historical use of the first device or the second device by a user.

Example 36 includes the method of any of examples 32-35, wherein calendaring the second instance is specific to the medication identifier.

Example 37 includes the method of any of examples 32-36, wherein the medication identifier is retrieved from an ontology.

Example 38 includes the method of any of examples 32-37, wherein a length of the sliding generation window is based on user input.

Example 39 includes a user device including means to perform one or more elements of a method described in or related to examples 32-38.

Example 40 includes a non-transitory computer-readable media including instructions to cause a user device, upon execution of the instructions by one or more processors of the user device, to perform one or more elements of a method described in or related to any of examples 32-38.

The various examples can be further implemented in a wide variety of operating environments, which in some cases can include one or more user computers, computing devices, or processing devices which can be used to operate any of a number of applications. User or client devices can include any of a number of general purpose personal computers, such as desktop or laptop computers running a standard operating system, as well as cellular, wireless, and handheld devices running mobile software and capable of supporting a number of networking and messaging protocols. Such a system also can include a number of workstations running any of a variety of commercially-available operating systems and other known applications for purposes such as development and database management. These devices also can include other electronic devices, such as dummy terminals, thin-clients, gaming systems, and other devices capable of communicating via a network.

While specific embodiments have been described, one skilled in the art will recognize that numerous modifications are possible. A single controller may use processes described herein to establish pairings with any number of accessories and to selectively communicate with different accessories at different times. Similarly, a single accessory may be controlled by multiple controllers with which it has established pairings. Any function of an accessory may be controlled by modeling the function as a service having one or more characteristics and allowing a controller to interact with (e.g., read, modify, receive updates) the service and/or its characteristics. Accordingly, protocols and communication processes as described herein may be “universal,” meaning that they may be applied in any context with one or more controllers and one or more accessories regardless of accessory function or controller form factor or specific interfaces.

Thus, although specific embodiments have been described, it will be appreciated that embodiments may include all modifications and equivalents within the scope of the following claims.

As described above, one aspect of the present technology is the gathering and use of data available from specific and legitimate sources to improve the delivery of messages from one device to one or more devices. The present disclosure contemplates that in some instances, this gathered data may include personal information data that uniquely identifies or may be used to identify a specific person. Such personal information data may include demographic data, location-based data, online identifiers, telephone numbers, email addresses, home addresses, data or records relating to a user's health or level of fitness (e.g., vital signs measurements, medication information, exercise information), date of birth, or any other personal information.

The present disclosure recognizes that the use of such personal information data, in the present technology, may be used to the benefit of users. For example, the personal information data may be used to deliver a command from a user profile on a computing device to one or more computing devices. Further, other uses for personal information data that benefit the user are also contemplated by the present disclosure. For instance, specific states of devices (e.g., medical care related devices, fitness devices, etc.) associated with the user may be transmitted from a device back to the user profile.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominent and easily accessible by users and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities may subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations that may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates embodiments in which users selectively block the use of, or access to, personal information data. That is, the present disclosure contemplates that hardware and/or software elements may be provided to prevent or block access to such personal information data. For example, such as in the case of advertisement delivery services, the present technology may be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

While specific embodiments have been described, one skilled in the art will recognize that numerous modifications are possible. A single controller may use processes described herein to establish pairings with any number of accessories and to selectively communicate with different accessories at different times. Similarly, a single accessory may be controlled by multiple controllers with which it has established pairings. Any function of an accessory may be controlled by modeling the function as a service having one or more characteristics and allowing a controller to interact with (e.g., read, modify, receive updates) the service and/or its characteristics. Accordingly, protocols and communication processes as described herein may be “universal,” meaning that they may be applied in any context with one or more controllers and one or more accessories regardless of accessory function or controller form factor or specific interfaces.

Thus, although specific embodiments have been described, it will be appreciated that embodiments may include all modifications and equivalents within the scope of the following claims.

As described above, one aspect of the present technology is the gathering and use of data available from specific and legitimate sources to improve the delivery of messages from one device to one or more devices. The present disclosure contemplates that in some instances, this gathered data may include personal information data that uniquely identifies or may be used to identify a specific person. Such personal information data may include demographic data, location-based data, online identifiers, telephone numbers, email addresses, home addresses, data or records relating to a user's health or level of fitness (e.g., vital signs measurements, medication information, exercise information), date of birth, or any other personal information.

The present disclosure recognizes that the use of such personal information data, in the present technology, may be used to the benefit of users. For example, the personal information data may be used to deliver a command from a user profile on a computing device to one or more computing devices. Further, other uses for personal information data that benefit the user are also contemplated by the present disclosure. For instance, specific states of devices (e.g., medical care related devices, fitness devices, etc.) associated with the user may be transmitted from a device back to the user profile.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominent and easily accessible by users and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities may subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations that may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates embodiments in which users selectively block the use of, or access to, personal information data. That is, the present disclosure contemplates that hardware and/or software elements may be provided to prevent or block access to such personal information data. For example, such as in the case of token generation services, the present technology may be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Illustrative techniques for using a computing devices to delegate authority to generate a token from an owner to a sharing platform and provisioning the token by the sharing platform. Some or all of these techniques may, but need not, be implemented at least partially by as those shown at least in FIGS. 1-12 above. While many of the embodiments are described above with reference to computing devices and computing devices, it should be understood that other types of computing devices may be suitable to perform the techniques disclosed herein. Further, in the foregoing description, various non-limiting examples were described. For purposes of explanation, specific configurations and details are set forth in order to provide a thorough understanding of the examples. However, it should also be apparent to one skilled in the art that the examples may be practiced without the specific details. Furthermore, well-known features were sometimes omitted or simplified in order not to obscure the example being described.

The various embodiments further can be implemented in a wide variety of operating environments, which in some cases can include one or more user computers, computing devices or processing devices that can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system also can include a number of workstations running any of a variety of commercially-available operating systems and other known applications for purposes such as development and database management. These devices also can include other electronic devices, such as dummy terminals, thin-clients, gaming systems and other devices capable of communicating via a network.

Most embodiments utilize at least one network that would be familiar to those skilled in the art for supporting communications using any of a variety of commercially-available protocols, such as TCP/IP, OSI, FTP, UPnP, NFS, CIFS, and AppleTalk. The network can be, for example, a local area network, a wide-area network, a virtual private network, the Internet, an intranet, an extranet, a public switched telephone network, an infrared network, a wireless network, and any combination thereof.

In embodiments utilizing a network server, the network server can run any of a variety of server or mid-tier applications, including HTTP servers, FTP servers, CGI servers, data servers, Java servers, and business application servers. The server(s) also may be capable of executing programs or scripts in response requests from computing devices, such as by executing one or more applications that may be implemented as one or more scripts or programs written in any programming language, such as Java®, C, C # or C++, or any scripting language, such as Perl, Python or TCL, as well as combinations thereof. The server(s) may also include database servers, including without limitation those commercially available from Oracle®, Microsoft®, Sybase®, and IBM®

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen or keypad), and at least one output device (e.g., a display device, printer or speaker). Such a system may also include one or more storage devices, such as disk drives, optical storage devices, and solid-state storage devices such as RAM or ROM, as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a non-transitory computer readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services or other elements located within at least one working memory device, including an operating system and application programs, such as a client application or browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Non-transitory storage media and computer-readable storage media for containing code, or portions of code, can include any appropriate media known or used in the art such as, but not limited to, volatile and non-volatile, removable and non-removable media implemented in any method or technology for storage of information such as computer-readable instructions, data structures, program modules or other data, including RAM, ROM, Electrically Erasable Programmable Read-Only Memory (EEPROM), flash memory or other memory technology, CD-ROM, DVD or other optical storage, magnetic cassettes, magnetic tape, magnetic disk storage or other magnetic storage devices or any other medium that can be used to store the desired information and that can be accessed by the a system device. Based at least in part on the disclosure and teachings provided herein, a person of ordinary skill in the art will appreciate other ways and/or methods to implement the various embodiments. However, computer-readable storage media does not include transitory media such as carrier waves or the like.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a,” “an,” and “the,” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. The phrase “based at least in part on” should be understood to be open-ended, and not limiting in any way, and is intended to be interpreted or otherwise read as “based at least in part on,” where appropriate. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present. Additionally, conjunctive language such as the phrase “at least one of X, Y, and Z,” unless specifically stated otherwise, should also be understood to mean X, Y, Z, or any combination thereof, including “X, Y, and/or Z.”

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1-30. (canceled)

31. A method, comprising:
receiving, by a user device, a first interval of days associated with a medication identifier and a second interval of days associated with the medication identifier, wherein the second interval of days sequentially follows the first interval of days;
determining, by the user device, a number of days of a medication period, the medication period comprising the first interval of days followed by the second interval of days;
generating, by the user device, a second instance of the medication identifier; and
calendaring, by the user device, the second instance on an electronic calendar via a sliding generation window, the sliding generation window comprising a consecutive set of days of the medication period.

32. The method of claim 31, wherein the first interval of days is designated for taking a first medication, and wherein the second interval of days is designated for refraining from taking the first medication.

33. The method of claim 31, wherein calendaring the second instance comprises:
creating the sliding generation window after an initial medication date;
determining whether a selected day of the sliding generation window falls within the first interval of days or the second interval of days; and
calendaring the second instance based on the determination of whether the selected day falls within the first interval of days or the second interval of days.

34. The method of claim 31, wherein the electronic calendar is synchronized between the user device and a second device, and the method further comprises displaying a notification on either the user device or second device based on a historical use of the user device or the second device by a user.

35. The method of claim 31, wherein calendaring the second instance is specific to the medication identifier.

36. The method of claim 31, wherein the medication identifier is retrieved from an ontology.

37. The method of claim 31, wherein a length of the sliding generation window is based on user input.

38. A user device, comprising:
a processor; and
a computer-readable medium comprising instructions stored thereon that, when executed by the processor, cause the processor to:
receive a first interval of days associated with a medication identifier and a second interval of days associated with the medication identifier, wherein the second interval of days sequentially follows the first interval of days;
determine a number of days of a medication period, the medication period comprising the first interval of days followed by the second interval of days;
generate a second instance of the medication identifier; and
calendar the second instance on an electronic calendar via a sliding generation window, the sliding generation window comprising a consecutive set of days of the medication period.

39. The user device of claim 38, wherein the first interval of days is designated for taking a first medication, and wherein the second interval of days is designated for refraining from taking the first medication.

40. The user device of claim 38, wherein calendaring the second instance comprises:
creating the sliding generation window after an initial medication date;
determining whether a selected day of the sliding generation window falls within the first interval of days or the second interval of days; and
calendaring the second instance based on the determination of whether the selected day falls within the first interval of days or the second interval of days.

41. The user device of claim 38, wherein the electronic calendar is synchronized between the user device and a second device, and wherein the instructions stored thereon that, when executed by the processor, cause the processor to display a notification on either the user device or the other device based on a historical use of the user device or the second device by a user.

42. The user device of claim 38, wherein calendaring the second instance is specific to the medication identifier.

43. The user device of claim 38, wherein the medication identifier is retrieved from an ontology.

44. The user device of claim 38, wherein a length of the sliding generation window is based on user input.

45. A non-transitory computer-readable medium comprising instructions stored thereon, that when executed by a processor, cause the processor to execute operations comprising:
receiving a first interval of days associated with a medication identifier and a second interval of days associated with the medication identifier, wherein the second interval of days sequentially follows the first interval of days;
determining a medication time interval, the medication time interval comprising the first interval of days and the second interval of days;
generating a second instance of the medication identifier; and
calendaring the second instance of the medication identifier on an electronic calendar via a sliding generation window, the sliding generation window comprising a consecutive set of days of the medication period.

46. The non-transitory computer-readable medium of claim 45, wherein the first interval of days is designated for taking a first medication, and wherein the second interval of days is designated for refraining from taking the first medication.

47. The non-transitory computer-readable medium of claim 45, wherein
calendaring the second instance comprises:
creating the sliding generation window after an initial medication date;
determining whether a selected day of the sliding generation window falls within the first interval of days or the second interval of days; and
calendaring the second instance based on the determination of whether the selected day falls within the first interval of days or the second interval of days.

48. The non-transitory computer-readable medium of claim 45, wherein the electronic calendar is synchronized between the user device and a second device, and the method further comprises displaying a notification on either the user device or second device based on a historical use of the user device or the second device by a user.

49. The non-transitory computer-readable medium of claim 45, wherein calendaring the second instance is specific to the medication identifier.

50. The non-transitory computer-readable medium of claim 45, wherein the medication identifier is retrieved from an ontology.
